(19) United States
(12) Patent Application Publication
(10) Pub. No.: US 2004/0192707 A1
(43) Pub. Date: Sep. 30, 2004

(54) Compounds and methods for the treatment or prevention of Flavivirus infections

(76) Inventors: Laval Chan Chun Kong, Kirkland (CA); Oswy Z. Pereira, Kirkland (CA); Nghe Nguyen-Ba, LaPrairie (CA); Ming-Qiang Zhang, Kirkland (CA); Sanjoy Kumar Das, Laval (CA); Carl Poisson, Montreal (CA); Thumkunta Jagadeeswar Reddy, Ville St. laurent (CA); Liliane Halab, Outremont (CA)

Correspondence Address: MILLEN, WHITE, ZELANO & BRANIGAN, PC, 2200 CLARENDON BLVD, SUITE 1400, ARLINGTON, VA, 22201, US

(21) Appl. No.: 10/730,273

(22) Filed: Dec. 9, 2003

Related U.S. Application Data
(60) Provisional application No. 60/432,019, filed on Dec. 10, 2002.

(51) Int. Cl. A61K 31/52; A61K 31/513; A61K 31/453; C07D 473/14; C 07D 4 9/14

(52) U.S. Cl. 514/263.22; 514/269; 514/326; 544/277; 544/310; 546/207

(57) ABSTRACT

Compounds represented by formula I:

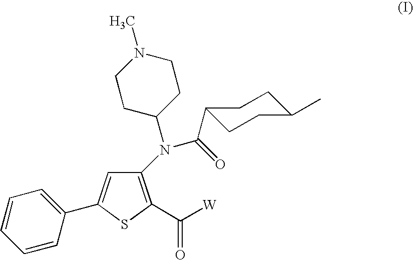

wherein W is as defined herein, and pharmaceutically acceptable salts thereof, are suitable for use in treating or preventing a Flaviviridae viral infection in a host.

This application claims the benefit of U.S. provisional application 60/432,019 filed Dec. 10, 2002 which is herein incorporated by reference.

### FIELD OF THE INVENTION

The present invention relates to novel compounds and a method for the treatment or prevention of Flavivirus infections using novel compounds.

### BACKGROUND OF THE INVENTION

Hepatitis is a disease occurring throughout the world. It is generally of viral nature, although there are other causes known. Viral hepatitis is by far the most common form of hepatitis. Nearly 750,000 Americans are affected by hepatitis each year, and out of those, more than 150,000 are infected with the hepatitis C virus (“HCV”).

HCV is a positive-stranded RNA virus belonging to the Flaviviridae family and has closest relationship to the pestiviruses that include hog cholera virus and bovine viral diarrhea virus (BVDV). HCV is believed to replicate through the production of a complementary negative-strand RNA template. Due to the lack of efficient culture replication system for the virus, HCV particles were isolated from pooled human plasma and shown, by electron microscopy, to have a diameter of about 50-60 nm. The HCV genome is a single-stranded, positive-sense RNA of about 9,600 bp coding for a polyprotein of 3009-3030 amino-acids, which is cleaved co and post-translationally by cellular and two viral proteinases into mature viral proteins (core, E1, E2, p7, NS2, NS3, NS4A, NS4B, NS5A, NS5B). It is believed that the structural proteins, E1 and E2, the major glycoproteins are embedded into a viral lipid envelope and form stable heterodimers. It is also believed that the structural core protein interacts with the viral RNA genome to form the nucleocapsid. The nonstructural proteins designated NS2 to NS5 include proteins with enzymatic functions involved in virus replication and protein processing including a polymerase, protease and helicase.

The main source of contamination with HCV is blood. The magnitude of the HCV infection as a health problem is illustrated by the prevalence among high-risk groups. For example, 60% to 90% of hemophiliacs and more than 80% of intravenous drug abusers in western countries are chronically infected with HCV. For intravenous drug abusers, the prevalence varies from about 28% to 70% depending on the population studied. The proportion of new HCV infections associated with post-transfusion has been markedly reduced lately due to advances in diagnostic tools used to screen blood donors.

The only treatment currently available for HCV infection is interferon-α (IFN-α). However, according to different clinical studies, only 70% of treated patients normalize alanine aminotransferase (ALT) levels in the serum and after discontinuation of IFN, 35% to 45% of these responders relapse. In general, only 20% to 25% of patients have long-term responses to IFN. Clinical studies have shown that combination treatment with IFN and ribavirin (RIBA) results in a superior clinical response than IFN alone. Different genotypes of HCV respond differently to IFN therapy, genotype 1b is more resistant to IFN therapy than type 2 and 3.

A prodrug is a chemical derivatives of a parent drug molecule that undergoes transformation within the body to release the parent drug. Prodrugs are generally recognized as being useful derivatives to improve several aspects of a parent drug such as low bioavailability, poor solubility in the GI tract or chemical instability in the GI tract. Detailed discussion on prodrugs may be found in “A Textbook of Drug Design and Development” by Povl Krogsgaard-Larsen & Hans Bundgaard,© 1991, Harwood Academic Publishers

There is therefore a great need for the development of efficacious anti-viral agents.

### SUMMARY OF THE INVENTION

In one aspect, the present invention provides novel compounds represented by formula I:

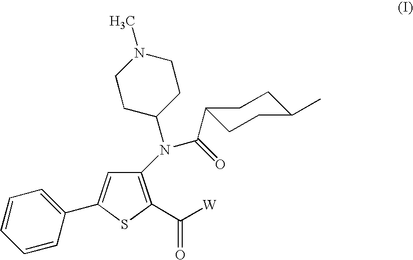

or pharmaceutically acceptable salts thereof;

wherein;

W is C₁₋₁₂ alkyloxy, C₆₋₁₂ arylalkyloxy, amino acid ester, nucleoside, C₆₋₁₂ heteroaralkyloxy, C₆ aryloxy, 5-6 membered heteroaryloxy,

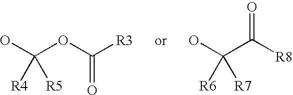

R₃ or R₈ is C₁₋₁₂ alkyl, C₆₋₁₀ aryl, C₆₋₁₀ arylalkyl, C₃₋₁₀ heterocycle, C₃₋₁₂ heteroaralkyl, C₆₋₁₂ aralkyl, C₁₋₁₂ alkyloxy or C₆₋₁₀ aryloxy or NR₁₀R₁₁ wherein

R₁₀ and R₁₁ are each independently H, C₁₋₁₂ alkyl, C₆₋₁₂ aryl, C₃₋₁₀ heterocycle, C₆₋₁₂ aralkyl or C₃₋₁₀ heteroaralkyl;

R₄ and R₅ are each independently chosen from H, C₁₋₁₂ alkyl, C₆₋₁₀ aryl, —O(CO)C₁₋₆ alkyl or C₃₋₁₀ heterocycle;

R₆ and R₇ are each independently chosen from H, C₁₋₁₂ alkyl, C₆₋₁₀ aryl, —O(CO)C₁₋₆ alkyl or C₃₋₁₀ heterocycle.

In another aspect, there is provided a method for treating or preventing a Flaviviridae viral infection in a host comprising administering to the subject a therapeutically effective amount of a compound, composition or combination of the invention.

In another aspect, there is provided a combination comprising a compound of the invention and one or more additionnal agent chosen from viral serine protease inhibitor, viral polymerase inhibitor and viral helicase inhibitor, immunomudulating agent, antioxydant agent, antibacterial agent or antisense agent.

In another aspect, there is provided a pharmaceutical composition comprising at least one compound of the invention and at least one pharmaceutically acceptable carrier or excipient.

In a further aspect, there is provided the use of compound, composition or combination of the invention for treating or preventing Flaviviridae viral infection in a host.

In still another aspect, there is provided the use of a compound of the invention for inhibiting or reducing the activity of viral polymerase in a host.

In still another aspect, there is provided the use of a compound of the invention for the manufacture of a medicament for treating or preventing a viral Flaviridae infection in a host.

In another aspect, there is provided a method of increasing oral bioavailability of an orally administrable compound of formula II

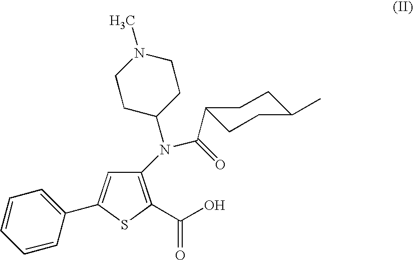

in a host comprising administering to said host a therapeutically effective amount of a compound of the present invention.

In still another aspect, there is provided a method of generating a compound of formula II:

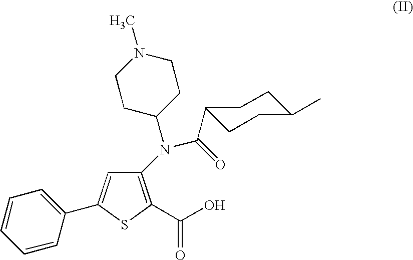

in a host, which comprises orally administering a compound of the present invention to said host.

### DETAILED DESCRIPTION OF THE INVENTION

In one embodiment, compounds of the present invention comprise those wherein the following embodiments are present, either independently or in combination.

In one embodiment, the present invention provides novel compounds represented by formula I:

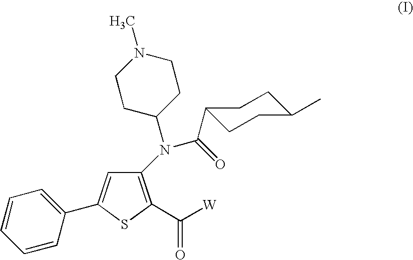

or pharmaceutically acceptable salts thereof.

In one embodiment, W is C₁₋₁₂ alkyloxy, C₆₋₁₂ arylalkyloxy, amino acid ester, nucleoside, C₆₋₁₂ heteroaralkyloxy, C6 aryloxy, 5-6 membered heteroaryloxy,

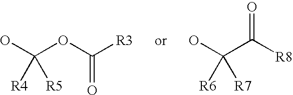

In further embodiments:

W is C₁₋₁₂ alkyloxy;

W is C₁₋₆ alkyloxy;

W is methoxy, ethyloxy, propyloxy, isopropyloxy, benzyloxy or 2′deoxycytidine-4-yl;

W is amino acid ester;

W is alanine methyl ester, valine methyl ester.

In one embodiment, W is

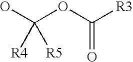

In one embodiment, W is

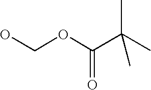

In a further embodiment, W is

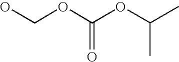

In one embodiment, W is

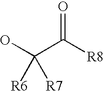

In a further embodiment, W is

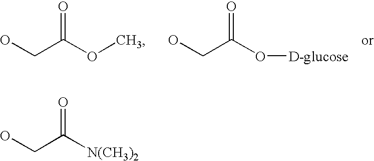

In one embodiment, R₃ or R₈ is C₁₋₁₂ alkyl, C₆₋₁₀ aryl, C₆₋₁₀ arylalkyl, C₃₋₁₀ heterocycle, C₃₋₁₂ heteroaralkyl, C₆₋₁₂ aralkyl, C₁₋₁₂ alkyloxy or C₆₋₁₀ aryloxy or NR₁₀R₁₁ wherein

R₁₀ and R₁₁ are each independently H, C₁₋₁₂ alkyl, C₆₋₁₂ aryl, C₃₋₁₀ heterocycle, C₆₋₁₂ aralkyl or C₃₋₁₀ heteroaralkyl.

In a further embodiment, R₃ or R₈ is C₁₋₆ alkyl, C₆₋₁₀ aryl, C₆₋₁₀ arylalkyl, C₃₋₆ heterocycle, C₃₋₁₀ heteroaralkyl, C₆₋₁₂ aralkyl, C₁₋₆ alkyloxy, C₆₋₁₀ aryloxy or NR₁₀R₁₁; wherein

R₁₀ and R₁₁ are each independently H, C₁₋₁₂ alkyl, C₆₋₁₂ aryl, C₃₋₁₀ heterocycle, C₆₋₁₂ aralkyl or C₃₋₁₀ heteroaralkyl.

In still a further embodiment, R₃ or R₈ is C₁₋₆ alkyl, C₆₋₁₀ aryl, C₁₋₆ alkyloxy, C₆₋₁₀ aryloxy or NR₁₀R₁₁; wherein

R₁₀ and R₁₁ are each independently H, C₁₋₆ alkyl.

In one embodiment, R₃ or R₈ is C₁₋₁₂ alkyl and is methyl, fluoromethyl, difluoromethyl, trifluoromethyl, ethyl, fluoroethyl difluoroethyl, trifluoroethyl, propyl, isopropyl, cyclopropyl, butyl, isobutyl, t-butyl, pentyl, cyclopentyl, hexyl, cyclohexyl.

In a further embodiment, R₃ or R₈ is C₁₋₁₂ alkyl and is ethyl, isopropyl, t-butyl or cyclohexyl.

In still a further embodiment, R₃ or R₈ is C₆₋₁₀ aryl and is phenyl.

In one embodiment, R₃ or R₈ is C₃₋₆ heterocycle.

In one embodiment, R₃ or R₈ is thienyl, furanyl, pyridyl, oxazolyl, thiazolyl, pyrrolyl, benzofuranyl, indolyl, benzoxazolyl, benzothienyl, benzothiazolyl, quinolinyl, pyridinyl, thiophenyl, benzofuran, thiazolyl, pyrazolyl, pyridinyl, isoxazolyl or tetrazolyl.

In further embodiments:

R₃ or R₈ is furanyl;

R₃ or R₈ is C₁₋₆ alkyloxy;

R₃ or R₈ is C₁₋₁₂ alkyloxy and is methoxy, ethyloxy, propyloxy, isopropyloxy, cyclopropyloxy or t-butyloxy;

R₃ or R₈ is C₆₋₁₀ aryloxy and is phenoxy.

In one embodiment, R₄ and R₅ are each independently H, C₁₋₁₂ alkyl, C₆₋₁₀ aryl, —O(CO)C₁₋₆ alkyl or C₃₋₁₀ heterocycle.

In one embodiment, R₄ and R₅ are each independently H, C₁₋₁₂ alkyl or C₆₋₁₀ aryl.

In further embodiments:

R₄ is H, C₁₋₁₂ alkyl or C₆₋₁₀ aryl and R₅ is H;

R₄ and R₅ are H;

R₄ is H;

R₄ and R₅ are C₁₋₁₂ alkyl;

R₄ is C₁₋₁₂ alkyl;

R₄ is C₁₋₆ alkyl and R₅ is H.

In one embodiment, R₄ is C₁₋₁₂ alkyl and is chosen from methyl, fluoromethyl, difluoromethyl, trifluoromethyl, ethyl, fluoroethyl difluoroethyl, trifluoroethyl, propyl, isopropyl, cyclopropyl, butyl, isobutyl, t-butyl, pentyl, cyclopentyl, hexyl or cyclohexyl.

In one embodiment, R₄ is C₁₋₁₂ alkyl and is chosen from methyl, trifluoromethyl, ethyl, trifluoroethyl, propyl, isopropyl, cyclopropyl, t-butyl or cyclohexyl.

In further embodiments:

R₄ is methyl;

R₄ is C₆₋₁₀ aryl;

R₄ is C₆ aryl;

R₄ is C₆₋₁₀ aryl and is phenyl.

In further embodiments:

R₅ is H;

R₅ is C₁₋₁₂ alkyl;

R₅ is C₁₋₆ alkyl.

In one embodiment, R₄ and R₅ are taken together to form a 3-6 membered cycloalkyl.

In further embodiments:

R₆ and R₇ are each independently H, C₁₋₁₂ alkyl, C₆₋₁₀ aryl, —O(CO)C₁₋₆ alkyl or C₃₋₁₀ heterocycle;

R₆ and R₇ are each independently H, C₁₋₁₂ alkyl or C₆₋₁₀ aryl;

R₆ is H, C₁₋₁₂ alkyl or C₆₋₁₀ aryl and R₇ is H;

R₆ and R₇ are H;

R₆ is H;

R₆ and R₇ are C₁₋₁₂ alkyl;

R₆ is C₁₋₁₂ alkyl;

R₆ is C₁₋₆ alkyl and R₇ is H.

In one embodiment, R₆ is C₁₋₁₂ alkyl and is chosen from methyl, fluoromethyl, difluoromethyl, trifluoromethyl, ethyl, fluoroethyl difluoroethyl, trifluoroethyl, propyl, isopropyl, cyclopropyl, butyl, isobutyl, t-butyl, pentyl, cyclopentyl, hexyl or cyclohexyl.

In one embodiment, R₆ is C₁₋₁₂ alkyl and is chosen from methyl, trifluoromethyl, ethyl, trifluoroethyl, propyl, isopropyl, cyclopropyl, t-butyl or cyclohexyl.

In further embodiments:

R₆ is methyl;

R₆ is C₆₋₁₀ aryl;

R₆ is C₆ aryl;

R₆ is C₆₋₁₀ aryl and is phenyl.

In further embodiments:

R₇ is H;

R₇ is C₁₋₁₂ alkyl;

R₇ is C₁₋₆ alkyl.

In one embodiment, R₆ and R₇ are taken together to form a 3-6 membered cycloalkyl.

In further embodiments:

R₁₀ and R₁₁ are each independently H, C₁₋₁₂ alkyl, C₃₋₁₀ heterocycle.

R₁₀ and R₁₁ are H;

R₁₀ and R₁₁ are C₁₋₁₂ alkyl;

R₁₀ is C₁₋₁₂ alkyl and R₁₁ is H;

R₁₀ is methyl, ethyl, propyl, isopropyl, butyl, terbutylhexyl or cyclohexyl and R₁₁ is H;

R₁₀ and R₁₁ are methyl, ethyl, propyl, isopropyl, butyl, terbutylhexyl or cyclohexyl.

In one aspect, the present invention provides novel compounds including:

Compound 1: 3-[(4-METHYL-CYCLOHEXANECARBONYL)-(1-METHYL-PIPERIDIN-4-YL)-AMINO]-5-PHENYL-THIOPHENE-2-CARBOXYLIC ACID 2,2-DIMETHYL-PROPIONYLOXYMETHY;

Compound 2: 4-[(2-ISOPROPOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM; CHLORIDE

Compound 3: 4-[(2-ISOPROPYLCARBAMOYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;

Compound 4: 1-METHYL-4-{(4-METHYL-CYCLOHEXANECARBONYL)-[2-(5-ETHYL-2-OXO-[1,3]DIOXOL-4-YLMETHOXYCARBONYL)-5-PHENYL-THIOPHEN-3-YL]-AMINO}-PIPERIDINIUM;

Compound 5: 4-[[2-(1-ISOPROPOXYCARBONYLOXY-ETHOXYCARBONYL)-5-PHENYL-THIOPHEN-3-YL]-(4-METHYL-CYCLOHEXANECARBONYL)-AMINO]-1-ETHYL-PIPERIDINIUM CHLORIDE;

Compound 6: 4-[{2-[1-(2,2-DIMETHYL-PROPIONYLOXY)-ETHOXYCARBONYL]-5-PHENYL-THIOPHEN-3-YL}-(4-METHYL-CYCLOHEXANECARBONYL)-AMINO]-1-ETHYL-PIPERIDINIUM CHLORIDE;

Compound 7: 4-[[2-(ISOPROPOXYCARBONYLOXY-PHENYL-METHOXYCARBONYL)-5-PHENYL-THIOPHEN-3-YL]-(4-METHYL-CYCLOHEXANECARBONYL)-AMINO]-1-ETHYL-PIPERIDINIUM CHLORIDE;

Compound 8: 4-[(2-CYCLOHEXYLOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;

Compound 9: 4-[{2-[(2,2-DIMETHYL-PROPIONYLOXY)-PHENYL-ETHOXYCARBONYL]-5-PHENYL-THIOPHEN-3-YL}-(4-METHYL-CYCLOHEXANECARBONYL)-AMINO]-1-METHYL-PIPERIDINIUM CHLORIDE;

Compound 10: 1-METHYL-4-[(4-METHYL-CYCLOHEXANECARBONYL)-(5-PHENYL-2-PROPIONYLOXYMETHOXYCARBONYL-THIOPHEN-3-YL)-AMINO]-PIPERIDINIUM; CHLORIDE;

Compound 11: 4-([2-(FURAN-2-CARBONYLOXYMETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;

Compound 12: 4-[(2-BENZOYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;

Compound 13: 4-[(2-CYCLOHEXANECARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;

Compound 14: 1-METHYL-4-[(4-METHYL-CYCLOHEXANECARBONYL)-(5-PHENYL-2-SUCCINYL-17(3-TERT-BUTOXYCARBONYLMETHYL-CARBAMOYL)-METHYL-PROPYL)-7,12-DIHYDROXY-10,13-DIMETHYL-HEXADECAHYDRO-CYCLOPENTA(A)PHENANTHREN-3-YLOYMETHOXYCARBONYL-THIOPHEN-3-YL)AMINO-PIPERIDINIUM CHLORIDE;

Compound 15: METHYL-4-[(4-METHYL-CYCLOHEXANECARBONYL)-(5-PHENYL-2-SUCCINYL-17(3-CARBONYLMETHYL-CARBAMOYL)-METHYL-PROPYL)-7,12-DIHYDROXY-10,13-DIMETHYL-HEXADECAHYDRO-CYCLOPENTA(A)PHENANTHREN-3-YLOYMETHOXYCARBONYL-THIOPHEN-3-YL)AMINO-PIPERIDINIUM CHLORIDE;

Compound 16: 4-[(2-ETHOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;

Compound 17: 1-METHYL-4-[(4-METHYL-CYCLOHEXANECARBONYL)-(2-PHENOXYCARBONYLOXYMETHOXYCARBONYL-5-PHENYL-THIOPHEN-3-YL)-AMINO]-PIPERIDINIUM CHLORIDE;

Compound 18: 1-METHYL-4-{(4-METHYL-CYCLOHEXANECARBONYL)-[2-(MORPHOLINE-4-CARBONYLOXYMETHOXYCARBONYL)-5-PHENYL-THIOPHEN-3-YL]-AMINO}-PIPERIDINIUM CHLORIDE;

Compound 19: 4-[{2-[1-(2,2-DIMETHYL-PROPIONYLOXY)-2-METHYL-PROPOXYCARBONYL]-5-PHENYL-THIOPHEN-3-YL}-(4-METHYL-CYCLOHEXANECARBONYL)-AMINO]-1-METHYL-PIPERIDINIUM CHLORIDE;

Compound 20: 4-[(2-SEC-BUTOXYCARBONYLOXYMETHOXYCARBONYL-5-PHENYL-THIOPHEN-3-YL)-(4-METHYL-CYCLOHEXANECARBONYL)-AMINO]-1-METHYL-PEPERIDINIUM CHLORIDE;

Compound 21: 4-[[2-(1-ISOPROPOXYCARBONYLOXY-2-METHYL-PROPOXYCARBONYL)-5-PHENYL-THIOPHEN-3-YL]-(4-METHYL-CYCLOHEXANECARBONYL)-AMINO]-1-METHYL-PIPERIDINIUM CHLORIDE;

Compound 22: 4-[(2-SEC-BUTOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM; CHLORIDE;

Compound 23: 3-[(4-METHYL-CYCLOHEXANECARBONYL)-(1-METHYL-PIPERIDIN-4-YL) -AMINO]-5-PHENYL-THIOPHENE-2-CARBOXYLIC ACID TERT-BUTOXYCARBONYLAMINOACETOXYMETHYL ESTER;

Compound 24: 3-[(4-METHYL-CYCLOHEXANECARBONYL)-(1-METHYL-PIPERIDIN-4-YL)-AMINO]-5-PHENYL-THIOPHENE-2-CARBOXYLIC ACID 2-TERT-BUTOXYCARBONYLAMINO-3-METHYL-BUTYRYLOXYMETHYL ESTER;

Compound 25: 3-[(4-METHYL-CYCLOHEXANECARBONYL)-(1-METHYL-PIPERIDIN-4-YL)-AMINO]-5-PHENYL-THIOPHENE-2-CARBOXYLIC ACID AMINOACETOXYMETHYL ESTER, BIS TRIFLUOROACETATE SALT;

Compound 26: 3-[(4-METHYL-CYCLOHEXANECARBONYL)-(1-METHYL-PIPERIDIN-4-YL)-AMINO]-5-PHENYL-THIOPHENE-2-CARBOXYLIC ACID 2-AMINO-3-METHYL-BUTYRYLOXYMETHYL ESTER, BIS TRIFLUOROACETATE SALT;

Compound 27: 4-[[2-(1-ISOPROPOXYCARBONYLOXY-1-METHYL-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;

Compound 28: 3-[(4-METHYL-CYCLOHEXANECARBONYL)-(1-METHYL-PIPERIDIN-4-YL)-AMINO]-5-PHENYL-THIOPHENE-2-CARBOXYLIC ACID 1-(1-METHYL-CYCLOHEXANECARBONYLOXY)-ETHYL ESTER;

Compound 29: 4-[[2-(1-#TERT!-BUTOXYCARBONYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;

Compound 30: 1-METHYL-4-((4-METHYL-CYCLOHEXANECARBONYL)-{2-[1-(1-METHYL-CYCLOHEXANECARBONYLOXY)-ETHOXYCARBONYL]-5-PHENYL-THIOPHEN-3-YL}-AMINO)-PIPERIDINIUM;

Compound 31: PYRROLIDINE-1,2-DICARBOXYLIC ACID 1-TERT-BUTYL ESTER 2-{3-[(4-METHYL-CYCLOHEXANECARBONYL)-(1-METHYL-PIPERIDIN-4-YL)-AMINO]-5-PHENYL-THIOPHENE-2-CARBONYLOXYMETHYL} ESTER;

Compound 32: 4-Methyl-piperazine-1-carboxylic acid 3-[(4-methyl-cyclohexanecarbonyl)-(1-methyl-piperidin-4-yl)-amino]-5-phenyl-thiophene-2-carbonyloxymethyl ester dihydrochloride salt;

Compound 33: 4-[[2-(1-CYCLOHEXYLOXYCARBONYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM; CHLORIDE;

Compound 34: 2-{3-[(4-METHYL-CYCLOHEXANECARBONYL)-(1-METHYL-PIPERIDIN-4-YL)-AMINO]-5-PHENYL-THIOPHENE-2-CARBONYLOXYMETHOXYCARBONYL}-PYRROLIDINIUM; BIS-TRIFLUORO-ACETATE;

Compound 35: 4-[[2-(1-ISOBUTYRYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM; CHLORIDE;

Compound 36: 3-[(4-METHYL-CYCLOHEXANECARBONYL)-(1-METHYL-PIPERIDIN-4-YL)-AMINO]-5-PHENYL-THIOPHENE-2-CARBOXYLIC ACID PYRIDIN-2-YL ESTER;

Compound 37: 4-[[2-(1-ACETOXY-1-METHYL-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;

Compound 38: 3-[(4-METHYL-CYCLOHEXANECARBONYL)-(1-METHYL-PIPERIDIN-4-YL)-AMINO]-5-PHENYL-THIOPHENE-2-CARBOXYLIC ACID 2-OXO-PYRROLIDIN-1-YLMETHYL ESTER;

Compound 39: 1-METHYL-4-[(4-METHYL-CYCLOHEXANECARBONYL)-(2-PHENOXYCARBONYL-5-PHENYL-THIOPHEN-3-YL)-AMINO]-PIPERIDINIUM CHLORIDE;

Compound 40: 1-METHYL-4-{(4-METHYL-CYCLOHEXANECARBONYL)-[5-PHENYL-2-(PYRIDIN-3-YLOXYCARBONYL)-THIOPHEN-3-YL]-AMINO}-PIPERIDINIUM; CHLORIDE;

Compound 41: 4-[{2-[1-(4-HYDROXY-5-HYDROXYMETHYL-TETRAHYDRO-FURAN-2-YL)-2-OXO-1,2-DIHYDRO-PYRIMIDIN-4-YLCARBAMOYL]-5-PHENYL-THIOPHEN-3-YL}-(4-METHYL-CYCLOHEXANECARBONYL)-AMINO]-1-METHYL-PIPERIDINIUM CHLORIDE;

Compound 42: 4-[[2-(1-METHOXYCARBONYL-2-METHYL-PROPYLCARBAMOYL)-5-PHENYL-THIOPHEN-3-YL]-(4-METHYL-CYCLOHEXANECARBONYL)-AMINO]-1-METHYL-PIPERIDINIUM CHLORIDE;

Compound 43: 4-[[2-(1-METHOXYCARBONYL-ETHYLCARBAMOYL)-5-PHENYL-THIOPHEN-3-YL]-(4-METHYL-CYCLOHEXANECARBONYL)-AMINO]-1-METHYL-PEPERIDINIUM;

or pharmaceutically acceptable salts thereof.

In one embodiment, the viral infection is chosen from Flavivirus infections.

In one embodiment, the Flavivirus infection is chosen from Hepatitis C virus (HCV), bovine viral diarrhea virus (BVDV), hog cholera virus, dengue fever virus, Japanese encephalitis virus and yellow fever virus.

In another embodiment, the Flavivirus infection is Hepatitis C viral infection.

In one embodiment, the present invention provides a method for treating or preventing a Flaviviridae viral infection in a host comprising administering to the host a therapeutically effective amount of at least one compound according to the invention described herein.

In one embodiment, the present invention provides a method for treating or preventing a Flaviviridae viral infection in a host comprising administering to the host a therapeutically effective amount of at least one compound according to the invention described herein and further comprising administering at least one additional agent chosen from viral serine protease inhibitor, viral polymerase inhibitor, viral helicase inhibitor, immunomudulating agent, antioxydant agent, antibacterial agent, therapeutic vaccine, hepatoprotectant agent or antisense agent.

In one embodiment, the additional agent is interferon α, ribavirin, silybum marianum, interleukine-12, amantadine, ribozyme, thymosin, N-acetyl cysteine or cyclosporin.

In one embodiment, the Flaviviridea viral infection is hepatitis C viral infection (HCV).

In one embodiment, the present invention provides a pharmaceutical composition comprising at least one compound according to the invention described herein and at least one pharmaceutically acceptable carrier or excipient.

In one embodiment, the present invention provides a pharmaceutical composition comprising at least one compound according to the invention described herein and at least one pharmaceutically acceptable carrier or excipient and further comprising at least one additional agent chosen from viral serine protease inhibitor, viral polymerase inhibitor, viral helicase inhibitor, immunomudulating agent, antioxydant agent, antibacterial agent, therapeutic vaccine, hepatoprotectant agent or antisense agent.

In another embodiment, the additional agent is interferon a, ribavirin, silybum marianum, interleukine-12, amantadine, ribozyme, thymosin, N-acetyl cysteine or cyclosporin.

In one embodiment, viral serine protease inhibitor is a flaviviridae serine protease inhibitor.

In one embodiment, viral polymerase inhibitor is a flaviviridae polymerase inhibitor.

In one embodiment, viral helicase inhibitor is a flaviviridae helicase inhibitor.

In further embodiments:

viral serine protease inhibitor is HCV serine protease inhibitor;

viral polymerase inhibitor is HCV polymerase inhibitor;

viral helicase inhibitor is HCV helicase inhibitor.

In one embodiment, there is provided a method for inhibiting or reducing the activity of viral polymerase in a host comprising administering a therapeutically effective amount of a compound according to the invention described herein.

In one embodiment, there is provided a method for inhibiting or reducing the activity of viral polymerase in a host comprising administering a therapeutically effective amount of a compound according to the invention described herein and further comprising administering one or more viral polymerase inhibitor.

In one embodiment, viral polymerase is a Flaviviridae viral polymerase.

In one embodiment, viral polymerase is a RNA-dependant RNA-polymerase.

In one embodiment, viral polymerase is HCV polymerase.

In one embodiment, there is provided a combination comprising a least one compound according to the invention described herein and one or more additional agent chosen from viral serine protease inhibitor, viral polymerase inhibitor and viral helicase inhibitor, immunomudulating agent, antioxydant agent, antibacterial agent, therapeutic vaccine, hepatoprotectant agent or antisense agent.

In one embodiment, the compound and additional agent are administered sequentially.

In one embodiment, the compound and additional agent are administered simultaneously.

The combinations referred to above may conveniently be presented for use in the form of a pharmaceutical formulation and thus pharmaceutical formulations comprising a combination as defined above together with a pharmaceutically acceptable carrier therefor comprise a further aspect of the invention.

The individual components for use in the method of the present invention or combinations of the present invention may be administered either sequentially or simultaneously in separate or combined pharmaceutical formulations.

In one embodiment, the present invention provides the use of a compound according to the invention described herein for treating or preventing Flaviviridae viral infection in a host.

In one embodiment, the present invention provides the use of a compound according to the invention described herein for the manufacture of a medicament for treating or preventing a viral Flaviridea infection in a host.

In one embodiment, the present invention provides the use of a compound according to the invention described herein for inhibiting or reducing the activity of viral polymerase in a host.

The combinations referred to above may conveniently be presented for use in the form of a pharmaceutical formulation and thus pharmaceutical formulations comprising a combination as defined above together with a pharmaceutically acceptable carrier therefor comprise a further aspect of the invention.

The individual components for use in the method of the present invention or combinations of the present invention may be administered either sequentially or simultaneously in separate or combined pharmaceutical formulations.

It will be appreciated by those skilled in the art that the compounds in accordance with the present invention can contain a chiral centre. The compounds of formula may thus exist in the form of two different optical isomers (i.e. (+) or (−) enantiomers). All such enantiomers and mixtures thereof including racemic mixtures are included within the scope of the invention. The single optical isomer or enantiomer can be obtained by method well known in the art, such as chiral HPLC, enzymatic resolution and chiral auxiliary.

Preferably, the compounds of the present invention are provided in the form of a single enantiomer at least 95%, more preferrably at least 97% and most preferably at least 99% free of the corresponding enantiomer.

More preferably the compound of the present invention are in the form of the (+) enantiomer at least 95% free of the corresponding (−) enantiomer.

More preferably the compound of the present invention are in the form of the (+) enantiomer at least 97% free of the corresponding (−) enantiomer.

More preferably the compound of the present invention are in the form of the (+) enantiomer at least 99% free of the corresponding (−) enantiomer.

In a more preferred embodiment, the compound of the present invention are in the form of the (−) enantiomer at least 95% free of the corresponding (+) enantiomer.

Most preferably the compound of the present invention are in the form of the (−) enantiomer at least 97% free of the corresponding (+) enantiomer.

More preferably the compound of the present invention are in the form of the (−) enantiomer at least 99% free of the corresponding (+) enantiomer.

It will also be appreciated that the compounds in accordance with the present invention can contain more than one chiral centres. The compounds may thus exist in the form of different diastereomers. All such diastereomers and mixtures thereof are included within the scope of the invention. The single diastereomer can be obtained by method well known in the art, such as HPLC, crystalisation and chromatography.

There is also provided a pharmaceutically acceptable salts of the compounds of the present invention. By the term pharmaceutically acceptable salts of compounds are meant those derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toleune-p-sulphonic, tartaric, acetic, trifluoroacetic, citric, methanesulphonic, formic, benzoic, malonic, naphthalene-2-sulphonic, cysteic acid and enzenesulphonic acids. Other acids such as oxalic, while not in themselves pharmaceutically acceptable, may be useful as intermediates in obtaining the compounds of the invention and their pharmaceutically acceptable acid addition salts.

Salts derived from appropriate bases include alkali metal (e.g. sodium), alkaline earth metal (e.g. magnesium), ammonium and NR₄+(where R is C₁₋₄ alkyl) salts.

References hereinafter to a compound according to the invention includes compounds and their pharmaceutically acceptable salts.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

As used in this application, the term “alkyl” represents a straight chain or branched chain hydrocarbon moiety which may optionally be substituted by one or more of halogen, nitro, nitroso, SO₃C₁₋₆ alkyl, SO₂C₁₋₆ alkyl, PO3RcRd, amido, C₁₋₆ alkyl, C₆₋₁₂ aralkyl, C₆₋₁₂ aryl, C₁₋₆ alkyloxy, C₆₋₁₂ aryloxy, C(O)C₁₋₆ alkyl, C(O)C₆₋₁₂ aryl, C(O)C₆₋₁₂ aralkyl, C₃₋₁₀ heterocycle, hydroxyl, amino, COOH, C(O)O—C₁₋₆ alkyl, cyano, azido, amidino or guanido;

wherein Rc and Rd are each independently chosen from H, C₁₋₆ alkyl or Rc and Rd are taken together with the oxygens to form a 5 to 10 membered heterocycle. Useful examples of alkyls include isopropyl, ethyl and hexyl. The term alkyl is also meant to include alkyls in which one or more hydrogen atoms is replaced by an halogen (e.g. CF3- or CF3CH2-). The term alkyl is also meant to include an alkyl containing at least one unsaturated group (e.g. allyl, acetylene, ethylene).

The term “cycloalkyl” represents a cyclic alkyl. The term cycloalkyl is also meant to include a cycloalkyl containing at least one unsaturated group. Useful examples of cycloalkyl include cyclopropyl, cyclobutyl, cyclohexenyl, cyclohex-dienyl and cyclohexyl.

The term “alkyloxy” represents an alkyl which is covalently bonded to the adjacent atom through an oxygen atom.

The term “amino” represents a basic organic compounds derived from ammonia (NH₃), in which one (primary amines), two (secondary amines), or three (tertiary amines) of the hydrogen atoms are replaced by organic radicals or groups. Useful examples of amino include alkylamines such as —NH(CH3), —N(CH3)2, —NH(propyl); arylamines such as —NH(phenyl); aralkylamine such as —NH(CH2-phenyl).

The term “amido” represents a compound formed from ammonia by replacement of one (or more than one) hydrogen atom by an acyl radical. Useful examples of amido include —CONH2, CONH(isopropyl), CON(CH3)2.

The term “aryl” represents a carbocyclic moiety containing at least one benzenoid-type ring which may optionally be substituted by one or more of halogen, nitro, nitroso, SO3C₁₋₆ alkyl, SO₂C₁₋₆ alkyl, PO₃RcRd, amido, C₁₋₆ alkyl, C₆₋₁₂ aralkyl, C₆₋₁₂ aryl, C₁₋₆ alkyloxy, C₆₋₁₂ aryloxy, C(O) C₁₋₆ alkyl, C(O)C₆₋₁₂ aryl, C(O)C₆₋₁₂ aralkyl, C₃₋₁₀ heterocycle, hydroxyl, amino, COOH, C(O)O—C₁₋₆ alkyl, cyano, azido, amidino or guanido;

wherein Rc and Rd are each independently chosen from H, C1-6 alkyl or Rc and Rd are taken together with the oxygens to form a 5 to 10 membered heterocycle. Examples of aryl include phenyl and naphthyl.

The term “aralkyl” represents an aryl group attached to the adjacent atom by a C₁₋₆ alkyl, C₁₋₆ alkenyl, or C₁₋₆ alkynyl (e.g., benzyl).

The term “aralkyloxy” represents an aralkyl which is covalently bonded to the adjacent atom through an oxygen atom

The term “amino acid ester” represents all the essential and non-essential alpha amino acids, beta amino acids and derivatives having the amino acid carboxylate esterified (e.g. isoleucine methyl ester, alanine ethyl ester, phenylglycine benzyl ester and beta-alanine phenyl ester). It will be understood that the amino acid ester may be linked by the alpha amino or any other suitable position of the amino acid ester to a carboxylic acid. Examples (arrows indicating examples of attachment points) include but are not limited to:

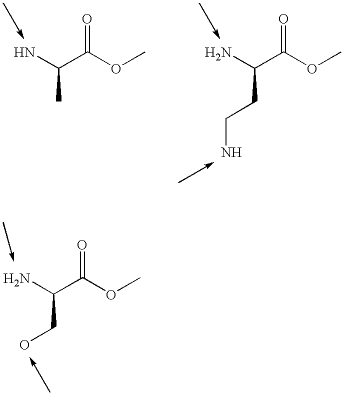

The term “heterocycle” represents a saturated or unsaturated, cyclic moiety wherein said cyclic moeity is interrupted by at least one heteroatom, (e.g. oxygen, sulfur or nitrogen) which may optionally be substituted by halogen, nitro, nitroso, SO₃C₁₋₆ alkyl, SO₂C₁₋₆ alkyl, PO₃RcRd, amido, C₁₋₆ alkyl, C₆₋₁₂ aralkyl, C₆₋₁₂ aryl, C₁₋₆ alkyloxy, C₆₋₁₂ aryloxy, C(O)C₁₋₆ alkyl, C(O)C₆₋₁₂ aryl, C(O)C₆₋₁₂ aralkyl, C₃₋₁₀ heterocycle, hydroxyl, amino, COOH, C(O)O—C₁₋₆ alkyl, cyano, azido, amidino or guanido;

wherein Rc and Rd are each independently chosen from H, C₁₋₆ alkyl or Rc and Rd are taken together with the oxygens to form a 5 to 10 membered heterocycle. Examples of heterocyclic rings include but are not limited to epoxide; furan; benzofuran; isobenzofuran; oxathiolane; dithiolane; dioxolane; pyrrole; pyrrolidine; imidazole; pyridine; pyrimidine; indole; piperidine; morpholine; thiophene and thiomorpholine.

The term “heteroaralkyl” represents an heterocycle group attached to the adjacent atom by a C₁₋₆ alkyl, C₁₋₆ alkenyl, or C₁₋₆ alkynyl.

As used in this application, the term “nucleoside” is meant to include natural and non natural nucleoside and their derivatives or analogues Such nucleoside, analogues and derivatives will be well known to those skilled in the art. It will be understood that the nucleoside may be linked by the base or the sugar portion to a carboxylic acid. Examples of suitable attachment points (indicated by arrows) include the following:

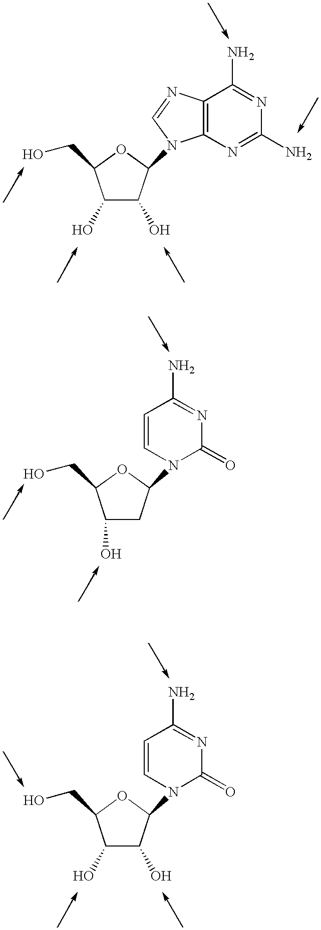

When there is a sulfur atom present, the sulfur atom can be at different oxidation levels, ie. S, SO, or SO2. All such oxidation levels are within the scope of the present invention.

When there is a nitrogen atom present, the nitrogen atom can be at different oxidation levels, ie. N or NO. All such oxidation levels are within the scope of the present invention.

The term “independently” means that a substituent can be the same or different definition for each item.

It will be appreciated that the amount of a compound of the invention required for use in treatment will vary not only with the particular compound selected but also with the route of administration, the nature of the condition for which treatment is required and the age and condition of the patient and will be ultimately at the discretion of the attendant physician or veterinarian. In general however a suitable dose will be in the range of from about 0.1 to about 750 mg/kg of body weight per day, preferably in the range of 0.5 to 60 mg/kg/day, most preferably in the range of 1 to 20 mg/kg/day.

The desired dose may conveniently be presented in a single dose or as divided dose administered at appropriate intervals, for example as two, three, four or more doses per day.

The compound is conveniently administered in unit dosage form; for example containing 10 to 1500 mg, conveniently 20 to 1000 mg, most conveniently 50 to 700 mg of active ingredient per unit dosage form.

Ideally the active ingredient should be administered to achieve peak plasma concentrations of the active compound of from about 1 to about 75 μM, preferably about 2 to 50 μM, most preferably about 3 to about 30 μM. This may be achieved, for example, by the intravenous injection of a 0.1 to 5% solution of the active ingredient, optionally in saline, or orally administered as a bolus containing about 1 to about 500 mg of the active ingredient. Desirable blood levels may be maintained by a continuous infusion to provide about 0.01 to about 5.0 g/kg/hour or by intermittent infusions containing about 0.4 to about 15 mg/kg of the active ingredient.

When the compounds of the present invention or a pharmaceutically acceptable salts thereof is used in combination with a second therapeutic agent active against the same virus the dose of each compound may be either the same as or differ from that when the compound is used alone. Appropriate doses will be readily appreciated by those skilled in the art.

While it is possible that, for use in therapy, a compound of the invention may be administered as the raw chemical it is preferable to present the active ingredient as a pharmaceutical composition. The invention thus further provides a pharmaceutical composition comprising compounds of the present invention or a pharmaceutically acceptable derivative thereof together with one or more pharmaceutically acceptable carriers therefor and, optionally, other therapeutic and/or prophylactic ingredients. The carrier(s) must be “acceptable” in the sense of being compatible with the other ingredients of the formulation and not deleterious to the recipient thereof. Pharmaceutical compositions include those suitable for oral, rectal, nasal, topical (including buccal and sub-lingual), transdermal, vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods include the step of bringing into association the active compound with liquid carriers or finely divided solid carriers or both and then, if necessary, shaping the product into the desired formulation.

Pharmaceutical compositions suitable for oral administration may conveniently be presented as discrete units such as capsules, cachets or tablets each containing a predetermined amount of the active ingredient; as a powder or granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing an/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use. For topical administration to the epidermis, the compounds according to the invention may be formulated as ointments, creams or lotions, or as a transdermal patch. Such transdermal patches may contain penetration enhancers such as linalool, carvacrol, thymol, citral, menthol and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

compositions suitable for topical administration in the mouth include lozenges comprising active ingredient in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the active compound with the softened or melted carrier(s) followed by chilling and shaping in moulds.

compositions suitable for vaginal administration may be presented as pessaries, tampons, creams, gels, pastes, foams or sprays containing in addition to the active ingredient such carriers as are known in the art to be appropriate.

For intra-nasal administration the compounds of the invention may be used as a liquid spray or dispersible powder or in the form of drops. Drops may be formulated with an aqueous or non-aqueous base also comprising one more dispersing agents, solubilising agents or suspending agents. Liquid sprays are conveniently delivered from pressurized packs.

For administration by inhalation the compounds according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

Alternatively, for administration by inhalation or insufflation, the compounds according to the invention may take the form of a dry powder composition, for example a powder mix of the compound and a suitable powder base such as lactose or starch. The powder composition may be presented in unit dosage form in, for example, capsules or cartridges or e.g. gelatin or blister packs from which the powder may be administered with the aid of an inhalator or insufflator.

When desired the above described formulations adapted to give sustained release of the active ingredient may be employed.

The following general schemes and examples are provided to illustrate various embodiments of the present invention and shall not be considered as limiting in scope.

### EXAMPLE 1

### 4-[(2-carboxy-5-phenyl-thiophen-3-yl)-(trans-4-methyl-cyclohexanecarbonyl)-amino]-1-methyl-piperidinium chloride

Step I

(a) To a stirred solution of 1-methyl-piperidin-4-one (6.0 g, 53 mmol, 6.52 mL) and Et₃N (14.16 g, 140 mmol, 19.5 mL) in 1,4-dioxane (20 mL) was added chlorotrimethylsilane (7.6 g, 70 mmol, 8.88 mL) drop wise during 30 min. The resultant reaction mixture was slowly heated to reflux at 110° C, stirred at the same temperature for 24 h, an additional amount of chlorotrimethylsilane (4.44 mL), heated for 24 h (take aliquot of it and run 1H NMR), cooled to room temp, filtered off the solid, solid was washed with n-pentane. The filtrate was concentrated on rotavaporator, and then diluted with n-pentane and filtered off the solid. The resultant solution was concentrated on rotavaporator followed by high vacuum furnished the 1-methyl-4-trimethylsilanyloxy-1,2,3,6-tetrahydro-pyridine (9.68 g, 1H NMR showed about 10:1 ratio of silylenolether and the starting material). The crude product was as such used in the next step without further purification.

(b) To a stirred solution of methyl-3-amino-5-phenylthiophene-carboxylate (233 mg, 1.0 mmol) and 1-methyl-4-trimethylsilanyloxy-1,2,3,6-tetrahydro-pyridine (370 mg, 2.0 mmol) in dichloroethane (3.0 mL) was added AcOH (0.114 mL, 2.0 eq) and followed addition of NaBH(OAc)₃ (424 mg, 2.0 mmol) in one portion. The resultant reaction mixture was stirred at RT for weekend, aq. 10% NaOH (until basic) was added, after 30 min, reaction mixture was extracted with dichloromethane. The organic extract was washed with brine and dried. The crude product was purified on silica gel column using 20% EtOAc/hexane for unreacted starting material followed by CHCl₃/MeOH/Et3N (180/16/1) furnished the 3-(1-methyl-piperidin-4-ylamino)-5-phenyl-thiophene-2-carboxylic acid methyl ester (240 mg, 73%). NMR ¹H (CDCl₃, 400 MHz): 7.64-7.6 (m, 2H), 7.43-7.34 (m, 3H), 6.83 (brs, 2H), 3.83 (s, 3H), 3.46-3.4 (m, 1H), 2.82-2.74 (m, 1H), 2.3 (s, 3H), 2.26-2.2 (m, 4H), 1.72-1.62 (m, 2H).

Step II

(a) To a stirred solution of trans-4-methylcyclohexyl acid (656 mg, 4.6 mmol) in dichloromethane (23 mL) was added a solution of oxalyl chloride (2 M, 4.6 mL) in dichloromethane followed by 2-3 drops of DMF (with 22 G needle), After stirred for 2 h, solvent and excess oxalyl chloride was removed on rotavaporator, trace amount of solvents removed under low vacuum (note: the product is very volatile, do not apply vacuum for long time, around 1-2 min). The crude 4-methyl-cyclohexanecarbonyl chloride was immediately used in the next step.

(b) To a stirred solution of the 3-(1-methyl-piperidin-4-ylamino)-5-phenyl-thiophene-2-carboxylic acid methyl ester (540 mg, 1.636 mmol) in 1,2-dichloroethane (15 mL) was added trans-4-methyl-cyclohexanecarbonyl chloride followed by PPh3 (429 mg, 1.635). The resultant reaction mixture was heated for 48 h at 90° C., cooled to room temperature, basified with aq. 10% NaOH solution, and then extracted with dichloromethane. The combined organic extract was washed with brine and dried, concentrated, purified on silica gel column chromatography using 200/90/16/1 (CH₂Cl₂/CHCl₃/MeOH/Et₃N) eluted first 3-[(trans-4-methyl-cyclohexanecarbonyl)-(1-methyl-piperidin-4-yl)-amino]-5-phenyl-thiophene-2-carboxylic acid methyl ester (760 mg, which contaminated with cyclohexyl acid) followed by starting material (270 mg). NMR¹H (CDCl₃, 400 MHz): 7.64-7.6 (m, 2H), 7.47-7.38 (m, 3H), 7.04 (s, 1H), 4.68-4.58 (m), 3.84 (s, 3H), 2.95-2.8 (m, 2H), 2.26 (s, 3H), 2.2-1.26 (m, 14H), 0.767 (d, J=6.6, 3H), 0.74-0.56 (m, 2H).

Step III

A mixture of 3-[(trans-4-methyl-cyclohexanecarbonyl)-(1-methyl-piperidin-4-yl)-amino]-5-phenyl-thiophene-2-carboxylic acid methyl ester (176 mg, 0.387 mmol) and LiOH.monohydrate (48.8 mg, 1.16 mmol, 4.0 eq) in dioxane:water (3:1, 3.9 mL, 0.1 M) was heated at 50° C. for 5 h, cooled to room temp, acidified with aq. 1N HCl, concentrated, diluted with small amount of water and filtered off the product, and then dried (136 mg), which was triturated with hexanes several times to remove 4-methylcyclohexylacid furnished 4-[(2-carboxy-5-phenyl-thiophen-3-yl)-(trans-4-methyl-cyclohexanecarbonyl)-amino]-1-methyl-piperidinium chloride, 101 mg, 60% yield).

NMR ¹H (CD₃OD, 400 MHz) : 7.76-7.72 (m, 2H), 7.5-7.38 (m, 4H) 4.8-4.65 (m, 1H), 3.6-3.4 (m, 2H), 3.25-3.2 (m, 2H), 2.8 (s, 3H), 2.3-1.2 (m, 12H), 0.78 (d, J=6.6 Hz, 3H), 0.96-0.58 (m, 2H).

### EXAMPLE 2

### 3-[(4-Methyl-cyclohexanecarbonyl)-(1-methyl-piperidin-4-yl)-amino]-5-phenyl-thiophene-2-carboxylic acid 2,2-dimethyl-propionyloxymethyl ester Compound 1.

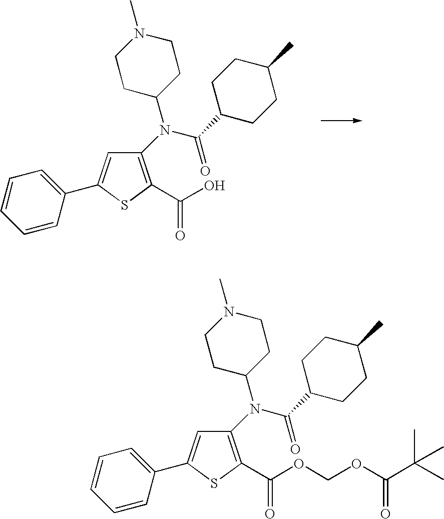

To a stirred solution of the 3-[(4-Methyl-cyclohexanecarbonyl)-(1-methyl-piperidin-4-yl)-amino]-5-phenyl-thiophene-2-carboxylic acid (5.00 g, 11.35 mmol) in DMF (75 ml) was added cesium carbonate (11.09 g, 34.04 mmol), sodium iodide (340 mg, 2.27 mmol) and chloromethyl pivalate (3.46 g, 22.70 mmol). The reaction mixture was stirred at room temperature for 3 hours and then evaporated to remove solvent. Water was added (50 ml) and the mixture was extracted with dichloromethane (3×75 ml). The combined extracts were then washed with brine (30 ml) and dried on Na₂SO₄, filtered and concentrated. The crude product was purified by flash chromatography (100% EtOAc), followed by trituration with hexane to give 3.26 g (52%) of 3-[(4-Methyl-cyclohexanecarbonyl)-(1-methyl-piperidin-4-yl)-amino]-5-phenyl-thiophene-2-carboxylic acid 2,2-dimethyl-propionyloxymethyl ester (Compound 1).

NMR ¹H (CDCl₃, 400 MHz): 7.65-7.62 ppm (m, 2H); 7.47-7.39 ppm (m, 3H); 7.07 ppm (s, 1H); 5.93 ppm (d, 1H); 5.84 ppm (d, 1H); 4.69-4.61 ppm (m, 1H); 2.95-2.82 ppm (m, 2H); 2.29 ppm (s, 3H); 2.22-2.11 ppm (m, 2H); 1.99-1.96 ppm (m, 2H); 1.77-1.57 ppm (m, 6H); 1.48-1.22 ppm (m, 4H); 1.21 ppm (s, 9H); 1,76 ppm (d, 3H); 0.69-0.56 ppm (m, 2H).

### EXAMPLE 3

### 3-[(4-Methyl-cyclohexanecarbonyl)-(1-methyl-piperidin-4-yl)-amino]-5-phenyl-thiophene-2-carboxylic acid isopropoxycarbonyloxymethyl ester Compound 2

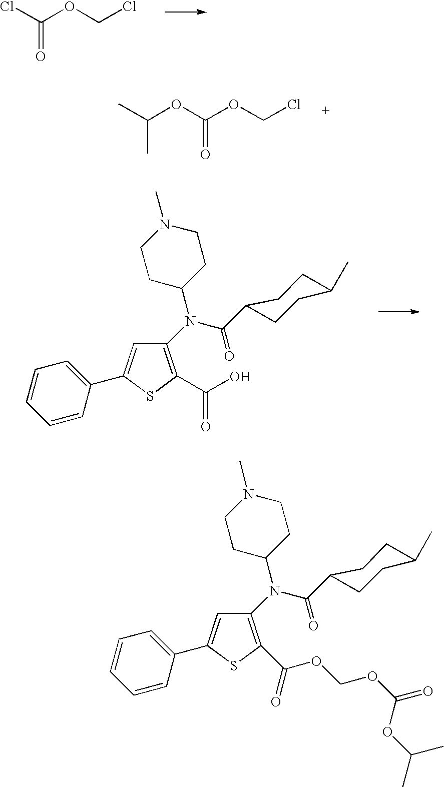

Step I

Carbonic Acid Chloromethyl Ester Isopropyl Ester*

A mixture of isopropanol (2.79 g, 46.5 mmol), triethylamine (4.94 g, 48.8 mmol) and dichloromethane (10.0 ml)was added drop wise to the stirred and cooled (0° C.) solution of chloromethyl choroformate (6.0 g, 46.5 mmol) in dichoromethane (30 ml) during 30 min. The mixture was stirred at this temperature for another 30 min then the precipitate was filtered off. The filtrate was washed twice with saturated NaHCO₃ solution and water. The solvent was evaporated and the residue purified by distillation to obtain 2.84 g (41%) of Carbonic acid chloromethyl ester isopropyl ester. NMR ¹H (CDCl₃, 400 MHz): δ 5.75 (s, 2H), 5.00 (m, 1H), 1.39 (d, 6H). *Procedure described in Synthetic communications, (1990) 20(18), pp2865-2885

Step II

3-[(4-Methyl-cyclohexanecarbonyl)-(1-methyl-piperidin-4-yl)-amino]-5-phenyl-thiophene-2-carboxylic acid isopropoxycarbonyloxymethyl ester

To a stirred solution of the 3-[(4-Methyl-cyclohexanecarbonyl)-(1-methyl-piperidin-4-yl)-amino]-5-phenyl-thiophene-2-carboxylic acid (1.00 g, 2.09 mmol) in DMF (15 ml) was added cesium carbonate (2.72 g, 8.36 mmol), sodium iodide (0.063 g, 0.42 mmol) and Carbonic acid chloromethyl ester isopropyl ester (0.639 g, 4.19 mmol). The reaction mixture was stirred at room temperature for 1 hours and then evaporated to remove solvent. Water was added (30 ml) and the mixture was extracted with dichloromethane (3×25 ml). The combined extracts were then washed with brine (30 ml) and dried on Na₂SO₄, filtered and concentrated. The crude product was purified by preparative tlc (5% MeOH/CH2Cl2) to give 0.230 mg (20%) of 3-[(4-Methyl-cyclohexanecarbonyl)-(1-methyl-piperidin-4-yl)-amino]-5-phenyl-thiophene-2-carboxylic acid isopropoxycarbonyloxymethyl ester (compound 2). NMR ¹H (CDCl₃, 400 MHz): δ 7.65 (d, 2H), 7.47 (m, 3H), 7.09 (s, 1H), 5.95 (dd, 2H), 4.95 (m, 1H), 4.65 (m, 1H), 2.89 (dd, 2H), 2.25 (s, 3H), 2.20-1.91 (m, 4H), 1.81-1.52 (m, 7H), 1.49-1.25 (m, 3H), 1.35 (m, 6H), 0.80 (d,3H), 0.79-0.56 (m, 2H)

### EXAMPLE 4

### 3-[(4-Methyl-cyclohexanecarbonyl)-(1-methyl-piperidin-4-yl)-amino]-5-phenyl-thiophene-2-carboxylic acid isopropoxycarbonyloxy-phenyl-methyl ester Compound 7.

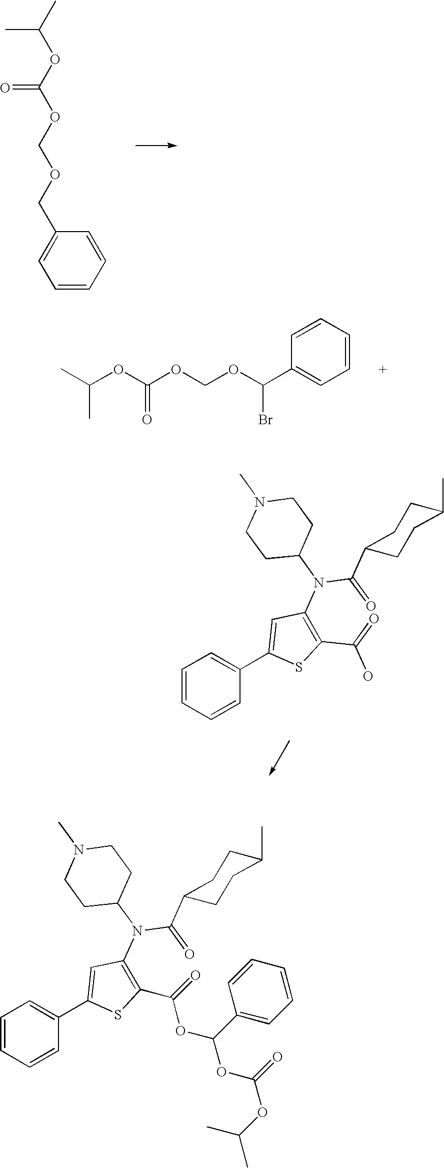

Step I

To a solution of Carbonic acid benzyloxymethyl ester isopropyl ester (0.235 g, 1.2 mmol) in CCl₄ (10 ml) was added NBS (0.260 g, 1.45 mmol) and AIBN (0.025 g). The reaction mixture was stirred for 1.5 h at reflux. The mixture was concentrated to one-half and filtered. The filtrated is evaporated to obtain 0.280 g (85%) of Carbonic acid bromo-phenyl-methoxymethyl ester isopropyl ester.

NMR ¹H (CDCl₃, 400 MHz): δ 7.61 (m, 3H), 7.45 (m, 3H), 5.05 (m, 1H), 1.42 (dd, 6H).

Step II

Esterification of 3-[(4-Methyl-cyclohexanecarbonyl)-(1-methyl-piperidin-4-yl)-amino]-5-phenyl-thiophene-2-carboxylic acid (0.24mmol, 120 mg) with Carbonic acid bromo-phenyl-methoxymethyl ester isopropyl ester (0.29 mmol, 80 mg), Cesium carbonate (195 mg, 0.6mmol), sodium Iodide (7 mg, 0.05 mmol) was carried out as described in example 2 and provided 3-[(4-Methyl-cyclohexanecarbonyl)-(1-methyl-piperidin-4-yl)-amino]-5-phenyl-thiophene-2-carboxylic acid isopropoxycarbonyloxy-phenyl-methyl ester (10%, 14 mg) (Compound 7);

NMR ¹H (CDCl₃, 400 MHz): δ 7.80-7.55 (m, 5H), 7.50-7.35 (m, 6H) 7.05(m, 1H), 4.95 (m, 1H), 4.62 (m, 1H), 2.95 (m, 2H), 2.75(m, 1H), 2.25 (s, 3H), 2.20-1.91 (m, 5H), 1.81-1.25 (m, 14H), 0.80 (d, 3H), 0.85-0.56 (m, 2H).

### EXAMPLE 5

### 1-Methyl-4-{(4-methyl-cyclohexanecarbonyl)-[5-phenyl-2-(pyridin-3-yloxycarbonyl)-thiophen-3-yl]-amino}-piperidinium chloride

Compound 40

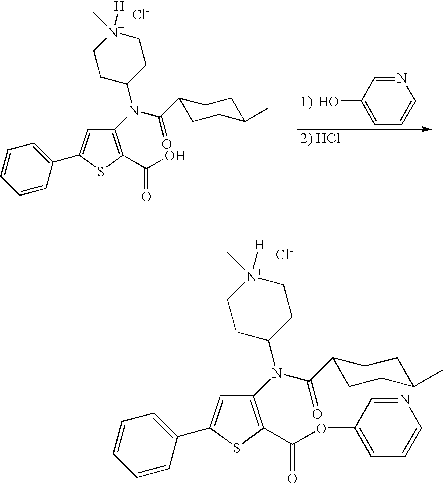

Step I

A solution of 4-[(2-Carboxy-5-phenyl-thiophen-3-yl)-(4-methyl-cyclohexanecarbonyl)-amino]-1-methyl-piperidinium chloride

(0.500 g, 1.05 mmol) in DMF (10 mL) was treated with Pyridin-3-ol (0.199 g, 2.1 mmol), EDC (0.402 g, 2.1 mmol) and DMAP (0.256 g, 2.1 mmol). The reaction was stirred at room temperature for 20 hours. EtOAc and NaHCO3 (aq) were added and the org layer was washed with water and brine, dried and evaporated to a residue that was purified by silica gel column chromatography using CH₂Cl₂:MeOH as eluent to provide 3-[(4-Methyl-cyclohexanecarbonyl)-(1-methyl-piperidin-4-yl)-amino]-5-phenyl-thiophene-2-carboxylic acid pyridin-3-yl ester as a white solid (0.324 g, 60% yield). ¹H NMR (CDCl₃, 300 MHz): 8.52 (m, 2H), 7.66 (m, 2H), 7.58 (m, 1H), 7.47 (m, 3H), 7.37 (dd, 1H), 7.13 (s, 1H), 4.68 (m, 1H), 2.85 (dd, 2H), 2.23 (s, 3H), 2.06 (m, 3H), 1.95 (d, 1H), 1.80 (d, 1H), 1.72-1.55 (m, 6H), 1.53-1.30 (m, 3H), 0.80 (d, 3H), 0.70 (m, 2H).

Step II

3-[(4-Methyl-cyclohexanecarbonyl)-(1-methyl-piperidin-4-yl)-amino]-5-phenyl-thiophene-2-carboxylic acid pyridin-3-yl ester (320 mg, 0.618 mmol) was dissolved in CH₂Cl₂ (6.2 mL) and treated with 4M solution of HCl in dioxane (0.23 mL, 0.93 mmol) at 0° C. The solution was stirred at room temperature for 15-30 min. The solvent was then evaporated to provide 1-Methyl-4-{(4-methyl-cyclohexanecarbonyl)-[5-phenyl-2-(pyridin-3-yloxycarbonyl)-thiophen-3-yl]-amino}-piperidinium; chloride as a pale yellow solid (0.300 g, 88% yield). ¹H NMR (CDCl₃, 300 MHz): 8.63 (d, 2H), 8.03 (d, 1H), 7.75 (m, 2H), 7.62 (m, 1H), 7.49 (m, 3H), 7.23 (s, 1H), 4.77 (m, 1H), 3.50 (d, 1H), 3.43 (d, 1H), 2.92 (m, 2H), 2.72 (d, 3H), 2.44 (m, 1 H), 2.30-2.15 (m, 2H), 1.70-1.56 (m, 5H), 1.50-1.25 (m, 2H), 0.78 (d, 3H), 0.70 (m, 2H).

The following compounds were prepared in a similar manner: Compound #36 and compound #39.

### EXAMPLE 6

### 4-[{2-[1-(4-Hydroxy-5-hydroxymethyl-tetrahydro-furan-2-yl)-2-oxo-1,2-dihydro-pyrimidin-4-ylcarbamoyl]-5-phenyl-thiophen-3-yl}-(4-methyl-cyclohexanecarbonyl)-amino]-1-methyl-piperidinium chloride; compound 41

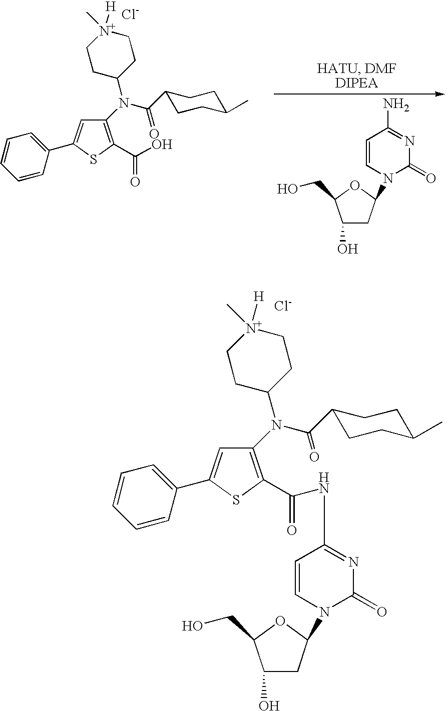

A solution of 4-[(2-Carboxy-5-phenyl-thiophen-3-yl)-(4-methyl-cyclohexanecarbonyl)-amino]-1-methyl-piperidinium chloride in DMF is treated with 2′-deoxy-cytidine (1.0 eq), HATU (1.1 eq), di-isopropylethylamine (2.0 eq) and DMAP (0.1 eq). The reaction is stirred at room temperature. EtOAc and NaHCO3 (aq) is added and the organic layer is washed with water and brine, dried and evaporated to a residue that is purified by silica gel column chromatography to provide the desired compound.

### EXAMPLE 7

### 4-[[2-(1-Methoxycarbonyl-2-methyl-propylcarbamoyl)-5-phenyl-thiophen-3-yl]-(4-methyl-cyclohexanecarbonyl)-amino]-1-methyl-piperidinium chloride; Compound 42

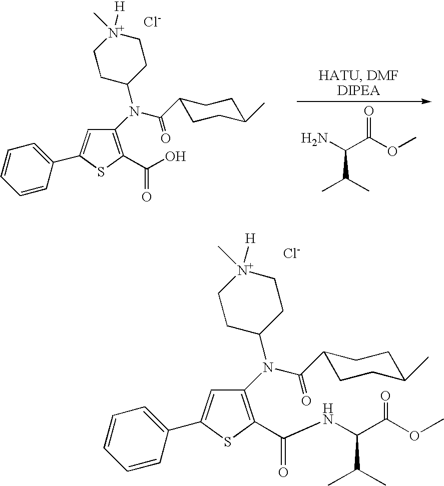

A solution of 4-[(2-Carboxy-5-phenyl-thiophen-3-yl)-(4-methyl-cyclohexanecarbonyl)-amino]-1-methyl-piperidinium chloride in DMF is treated with valine methyl ester (1.0 eq), HATU (1.1 eq), di-isopropylethylamine (2.0 eq) and DMAP (0.1 eq). The reaction is stirred at room temperature. EtOAc and NaHCO3 (aq) is added and the organic layer is washed with water and brine, dried and evaporated to a residue that is purified by silica gel column chromatography to provide the desired compound. Compound 43 is prepared similarly.

### EXAMPLE 8

### List of Compounds

TABLE 1

| Compound # | STRUCTURE | NAME |
|---|---|---|
| 1 | 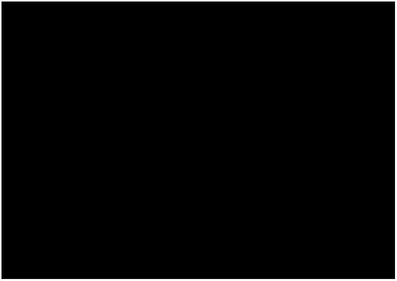 | 3-[(4-METHYL-CYCLOHEXANECARBONYL)-(1-METH- YL-PIPERIDIN-4-YL)-AMINO]-5-PHENYL-THIO- PHENE-2-CARBOXYLIC ACID 2,2-DI- METHYL-PROPIONYLOXYMETHY |
| 2 | 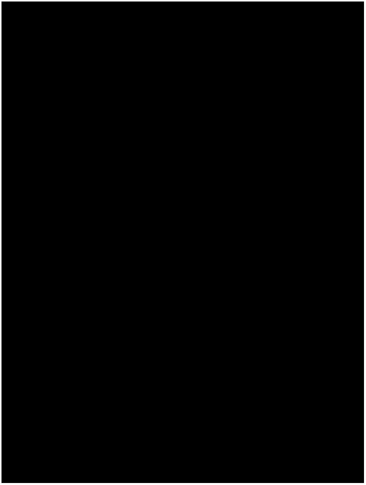 | 4-[(2-ISO- PROPOXYCARBONYLOXYMETHOXYCARBONYL-5-PHE- NYL-THIOPHEN-3-YL)-(4-METHYL-CYCLO- HEXANECARBONYL)-AMINO]-1-METHYL- PIPERIDINIUM; CHLORIDE |
| 4 | 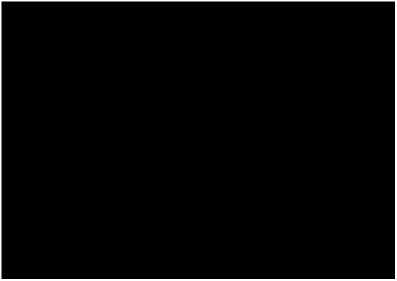 | 1-METHYL-4-{(4-METHYL- CYCLOHEXANECARBONYL)-[2-(5-METHYL-2-OXO-[1,3]DI- OXOL-4-YLMETHOXYCARBONYL)-5-PHE- NYL-THIOPHEN-3-YL]-AMINO}-PIPE- RIDINIUM |
| 5 | 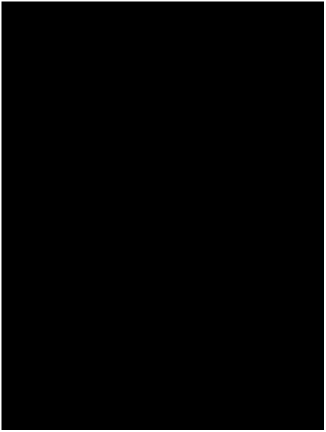 | 4-[[2-(1-ISOPROPOXYCARBONYLOXY-ETHOXY- CARBONYL)-5-PHENYL-THIOPHEN-3-YL]-(4-METH- YL-CYCLOHEXANECARBONYL)-AMINO]-1-METH- YL-PIPERIDINIUM; CHLORIDE |
| 6 | 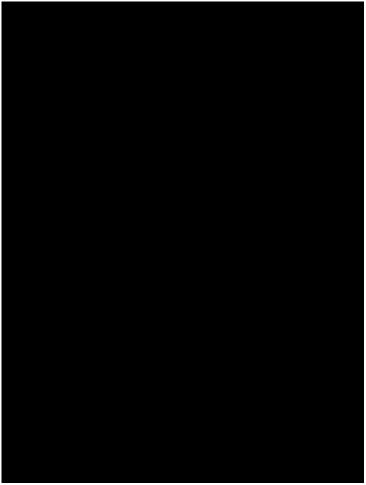 | 4-[{2-[1-(2,2-DIMETHYL- PROPIONYLOXY)-ETHOXYCARBONYL]-5-PHE- NYL-THIOPHEN-3-YL}-(4-METHYL- CYCLOHEXANECARBONYL)-AMINO]-1-METH- YL-PIPERIDINIUM CHLORIDE |
| 7 | 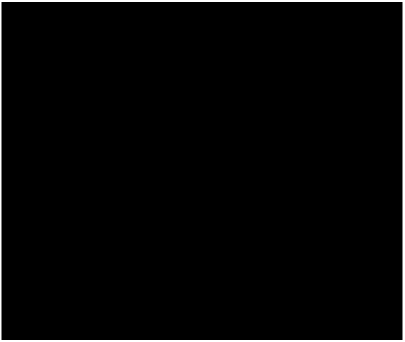 | 4-[[2-(ISOPROPOXYCARBONYLOXY-PHENYL- METHOXYCARBONYL)-5-PHENYL-THIOPHEN-3-YL]-(4-METH- YL-CYCLOHEXANECARBONYL)-A- MINO]-1-METHYL-PIPERIDINIUM; CHLORIDE |
| 8 | 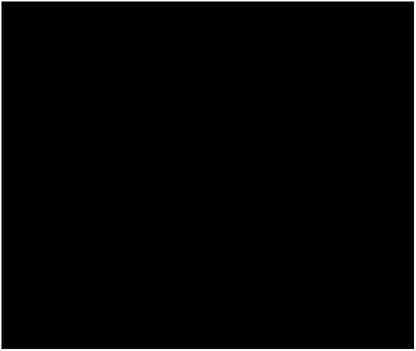 | 4-[(2-CYCLO- HEXYLOXYCARBONYLOXYMETHOXYCARBONYL-5-PHE- NYL-THIOPHEN-3-YL)-(4-METH- YL-CYCLOHEXANECARBONYL)-AMINO]-1-METH- YL-PIPERIDINIUM; CHLORIDE |
| 9 | 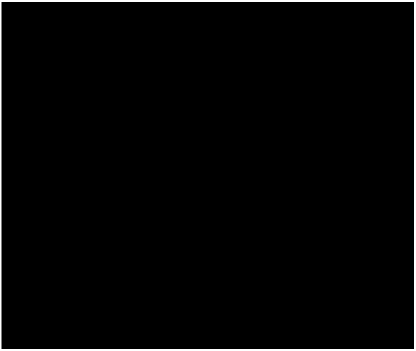 | 4-[{2-[(2,2-DIMETHYL-PROPIONYLOXY)-PHE- NYL-METHOXYCARBONYL]-5-PHENYL- THIOPHEN-3-YL}-(4-METHYL- CYCLOHEXANECARBONYL)-AMINO]-1-METHYL-PIPE- RIDINIUM; CHLORIDE |
| 10 | 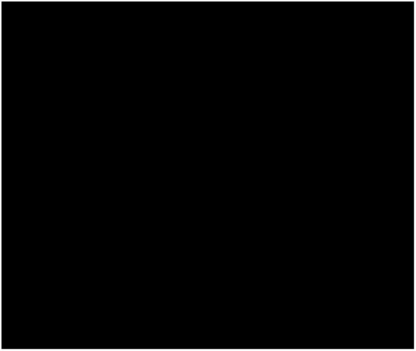 | 1-METHYL-4-[(4-METHYL- CYCLOHEXANECARBONYL)-(5-PHENYL-2-PRO- PIONYLOXYMETHOXYCARBONYL- THIOPHEN-3-YL)-AMINO]-PIPERIDINIUM; CHLORIDE |
| 11 | 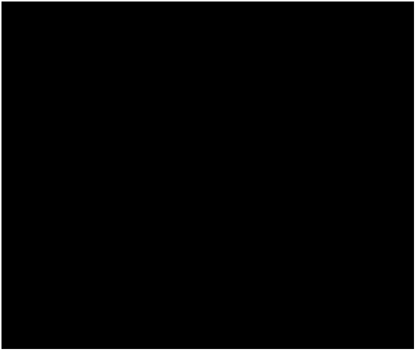 | 4-[[2-(FURAN-2-CARBONYL- OXYMETHOXYCARBONYL)-5-PHENYL-THIO- PHEN-3-YL]-(4-METHYL- CYCLOHEXANECARBONYL)-AMINO]-1-METHYL- PIPERIDINIUM; CHLORIDE |
| 12 | 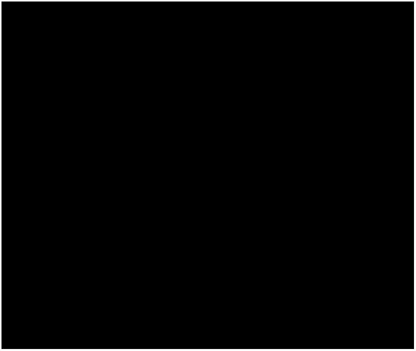 | 4-[(2-BENZOYLOXYMETHOXYCARBONYL-5-PHE- NYL-THIOPHEN-3-YL)-(4-METHYL- CYCLOHEXANECARBONYL)-AMINO]-1-METHYL- PIPERIDINIUM; CHLORIDE |
| 13 | 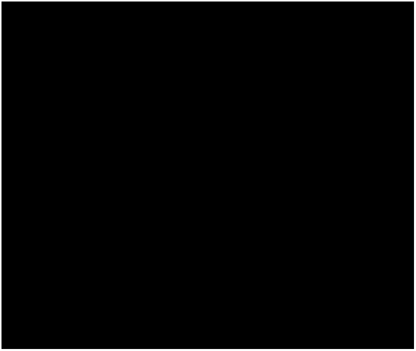 | 4-[(2-CYCLO- HEXANECARBONYLOXYMETHOXYCARBONYL-5-PHE- NYL-THIOPHEN-3-YL)-(4-METHYL- CYCLOHEXANECARBONYL)-AMINO]-1-METHYL- PIPERIDINIUM; CHLORIDE |
| 14 | 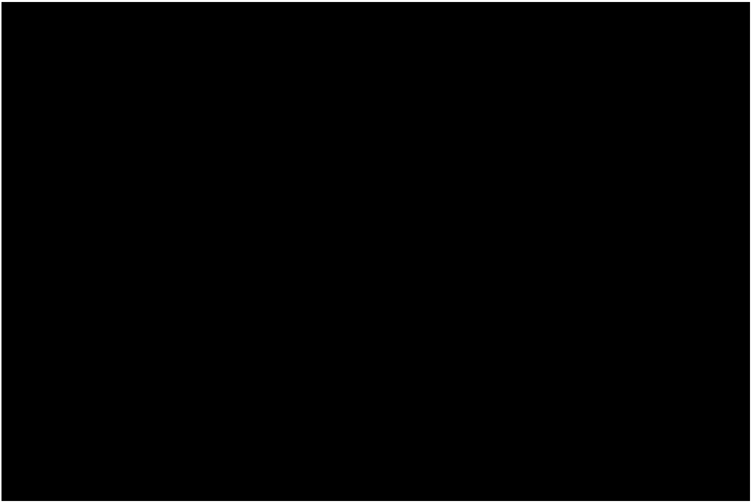 | 1-METHYL-4-{(4-METHYL- CYCLOHEXANECARBONYL)-(5-PHENYL-2-SUC- CINYL-17(3-TERT-BUTOXYCARBONYLMETHYL- CARBAMOYL)-METHYL-PROPYL)-7,12-DI- HYDROXY-10,13-DIMETHYL-HEXADECAHYDRO- CYCLOPENTA(A)PHENANTHREN-3-YLOYMETH- OXYCARBONYL-THIOPHEN-3-YL)AMINO- PIPERIDINIUM CHLORIDE |
| 15 | 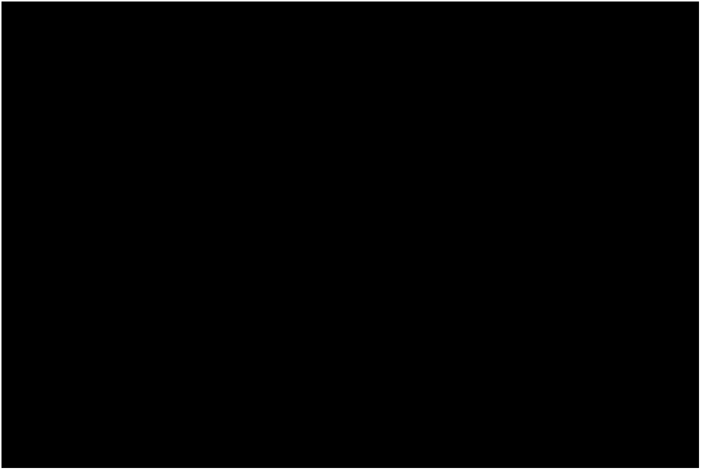 | METHYL-4-[(4-METHYL- CYCLOHEXANECARBONYL)-(5-PHENYL-2-SUC- CINYL-17(3-CARBONYLMETHYL-CARBAMOYL)-METH- YL-PROPYL)-7,12-DIHYDROXY-10,13-DI- METHYL-HEXADECAHYDRO- CYCLOPENTA(A)PHENANTHREN-3-YLOYMETH- OXYCARBONYL-THIOPHEN-3-YL)AMINO- PIPERIDINIUM CHLORIDE |
| 16 | 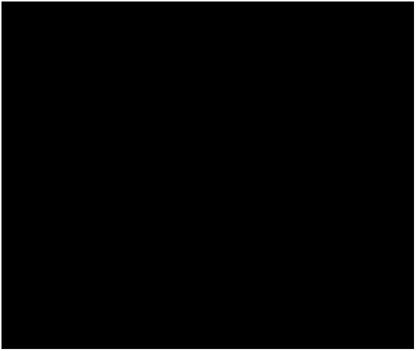 | 4-[(2-ETHOXYCARBONYLOXYMETHOXYCARBONYL-5-PHE- NYL-THIOPHEN-3-YL)-(4-METH- YL-CYCLOHEXANECARBONYL)-AMINO]-1-METHYL- PIPERIDINIUM; CHLORIDE |
| 17 | 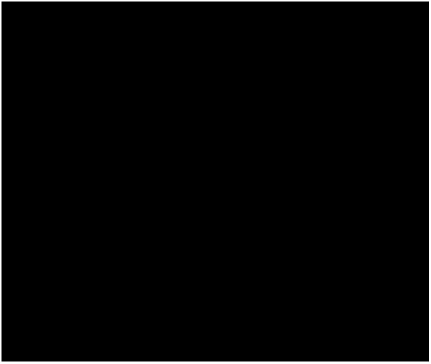 | 1-METHYL-4-[(4-METHYL- CYCLOHEXANECARBONYL)-(2-PHE- NOXYCARBONYLOXYMETHOXY- CARBONYL-5-PHENYL-THIOPHEN-3-YL)-A- MINO]-PIPERIDINIUM; CHLORIDE |
| 18 | 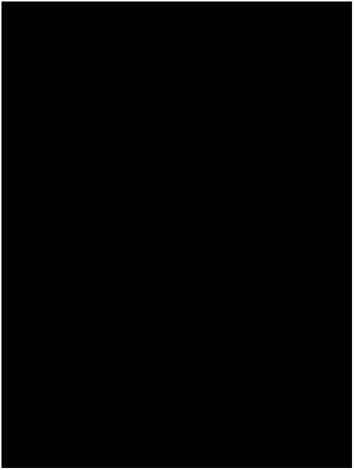 | 1-METHYL-4-{(4-METHYL- CYCLOHEXANECARBONYL)-[2-(MORPHOLINE-4-CARBO- NYLOXYMETHOXYCARBONYL)-5-PHENYL-THIO- PHEN-3-YL]-AMINO}-PIPERIDINIUM; CHLORIDE |
| 19 | 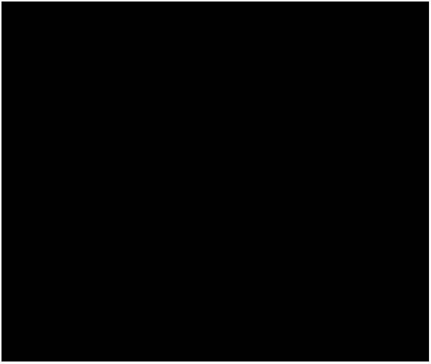 | 4-[{2-[1-(2,2-DIMETHYL-PROPIONYLOXY)-2-METH- YL-PROPOXYCARBONYL]-5-PHENYL- THIOPHEN-3-YL}-(4-METHYL- CYCLOHEXANECARBONYL)-AMINO]-1-METHYL- PIPERIDINIUM; CHLORIDE |
| 20 | 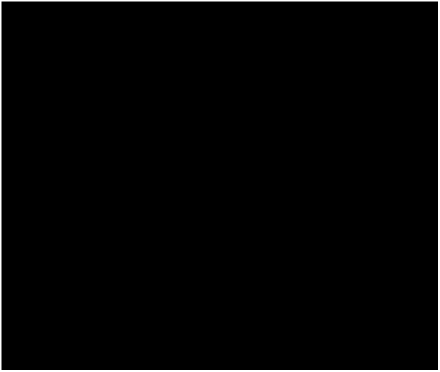 | 4-[(2-SEC- BUTOXYCARBONYLOXYMETHOXYCARBONYL-5-PHE- NYL-THIOPHEN-3-YL)-(4-METH- YL-CYCLOHEXANECARBONYL)-AMINO]-1-METH- YL-PIPERIDINIUM; CHLORIDE |
| 21 | 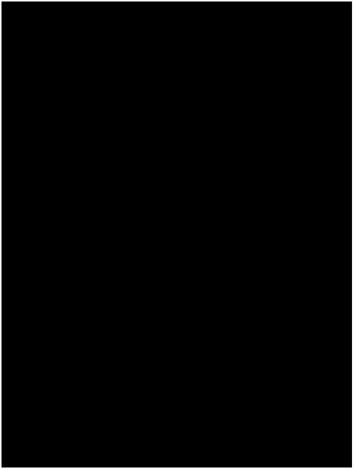 | 4-[[2-(1-ISOPROPOXYCARBONYLOXY-2-METHYL- PROPOXYCARBONYL)-5-PHENYL-THIOPHEN-3-YL]-(4-METH- YL-CYCLOHEXANECARBONYL)- AMINO]-1-METHYL-PIPERIDINIUM; CHLORIDE |
| 22 | 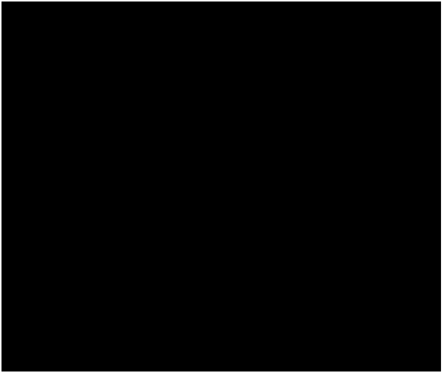 | 4-[(2-SEC- BUTOXYCARBONYLOXYMETHOXYCARBONYL-5-PHE- NYL-THIOPHEN-3-YL)-(4-METH- YL-CYCLOHEXANECARBONYL)-AMINO]-1-METH- YL-PIPERIDINIUM; CHLORIDE |
| 23 | 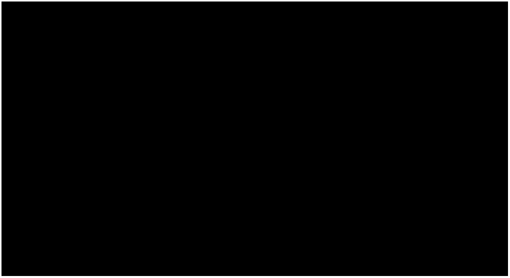 | 3-[(4-METHYL-CYCLOHEXANECARBONYL)-(1-METH- YL-PIPERIDIN-4-YL)-AMINO]-5-PHENYL- THIOPHENE-2-CARBOXYLIC ACID #TERT!-BU- TOXYCARBONYLAMINOACETOXYMETHYL ESTER |
| 24 | 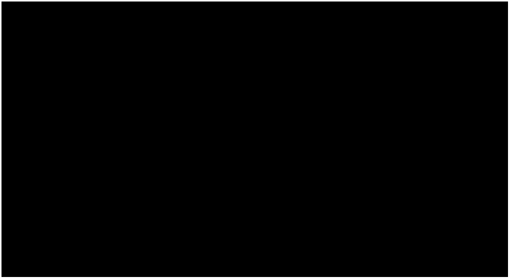 | 3-[(4-METHYL-CYCLOHEXANECARBONYL)-(1-METH- YL-PIPERIDIN-4-YL)-AMINO]-5-PHENYL- THIOPHENE-2-CARBOXYLIC ACID 2-#TERT!-BU- TOXYCARBONYLAMINO-3-METHYL- BUTYRYLOXYMETHYL ESTER |
| 25 | 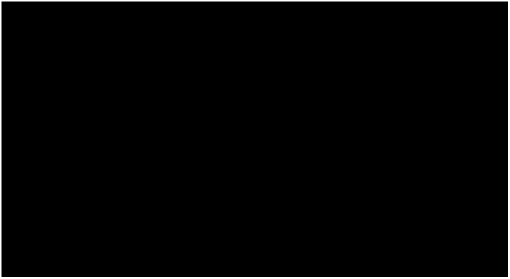 | 3-[(4-METHYL-CYCLOHEXANECARBONYL)-(1-METH- YL-PIPERIDIN-4-YL)-AMINO]-5-PHENYL- THIOPHENE-2-CARBOXYLIC ACID AMINOACETOXYMETHYL ESTER, BIS TRIFLUOROACETATE SALT |
| 26 | 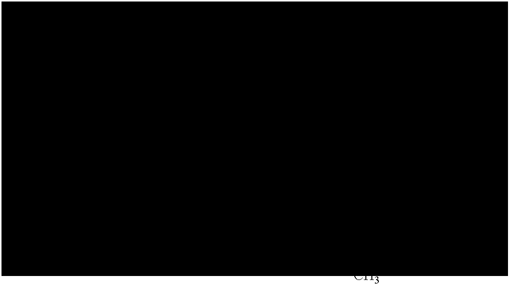 | 3-[(4-METHYL-CYCLOHEXANECARBONYL)-(1-METH- YL-PIPERIDIN-4-YL)-AMINO]-5-PHENYL- THIOPHENE-2-CARBOXYLIC ACID 2-AMINO-3-METH- YL-BUTYRYLOXYMETHYL ESTER, BIS TRIFLUOROACETATE SALT |
| 27 | 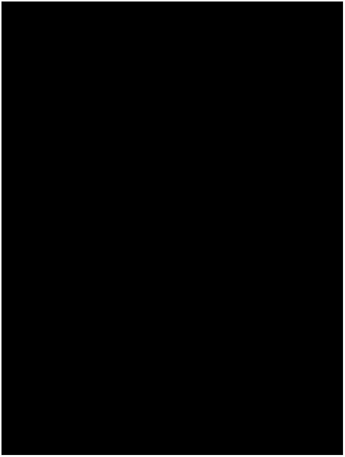 | 4-[[2-(1-ISOPROPOXYCARBONYLOXY-1-METHYL- ETHOXYCARBONYL)-5-PHENYL-THIOPHEN-3-YL]-(4-METH- YL-CYCLOHEXANECARBONYL)-AMINO]-1-METHYL- PIPERIDINIUM; CHLORIDE |
| 28 | 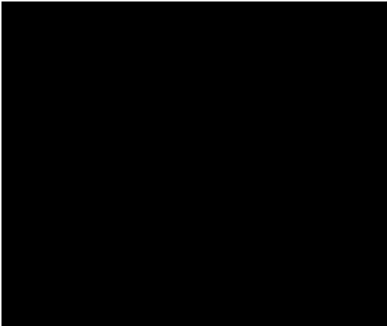 | 3-[(4-METHYL-CYCLOHEXANECARBONYL)-(1-METH- YL-PIPERIDIN-4-YL)-AMINO]-5-PHENYL- THIOPHENE-2-CARBOXYLIC ACID 1-(1-METHYL- CYCLOHEXANECARBONYLOXY)-ETHYL ESTER |
| 29 | 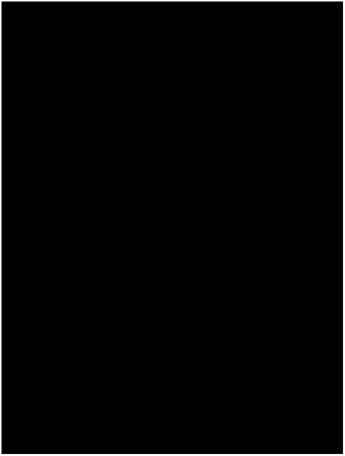 | 4-[[2-(1-TERT-BUTOXYCARBONYLOXY- ETHOXYCARBONYL)-5-PHENYL-THIOPHEN-3-YL]-(4-METH- YL-CYCLOHEXANECARBONYL)-AMINO]-1-METH- YL-PIPERIDINIUM; CHLORIDE |
| 30 | 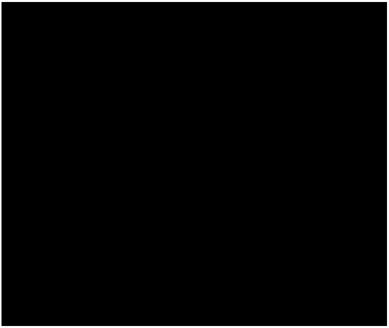 | 1-METHYL-4-((4-METHYL-CYCLO- HEXANECARBONYL)-{2-[1-(1-METH- YL-CYCLOHEXANECARBONYLOXY)-ETH- OXYCARBONYL]-5-PHENYL-THIOPHEN-3-YL}-A- MINO)-PIPERIDINIUM |
| 31 | 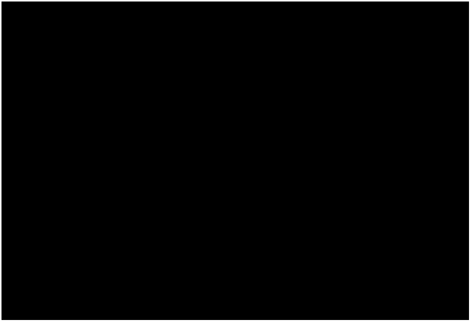 | PYRROLIDINE-1,2-DICARBOXYLIC ACID 1-#TERT!-BUTYL ESTER 2-{3-[(4-METHYL- CYCLOHEXANECARBONYL)-(1-METHYL-PIPERIDIN-4-YL)-A- MINO]-5-PHENYL-THIOPHENE-2-CAR- BONYLOXYMETHYL}ESTER |
| 32 | 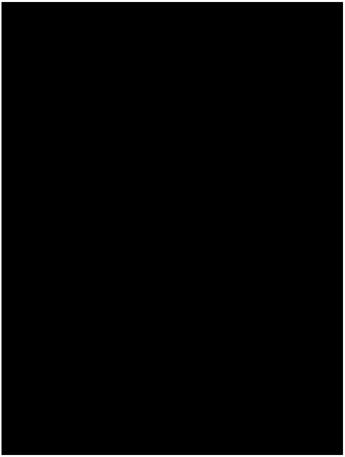 | 4-Methyl-piperazine-1-carboxylic acid 3-[(4-methyl- cyclohexanecarbonyl)-(1-methyl-piperidin-4-yl)-amino]-5-phe- nyl-thiophene-2-carbonyloxymethyl ester dihydrochloride salt |
| 33 | 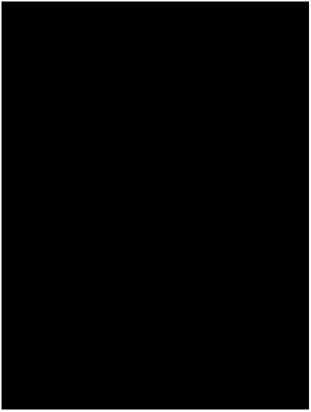 | 4-[[2-(1-CYCLOHEXYLOXYCARBONYLOXY- ETHOXYCARBONYL)-5-PHENYL-THIOPHEN-3-YL]-(4-METH- YL-CYCLOHEXANECARBONYL)-AMINO]-1-METH- YL-PIPERIDINIUM; CHLORIDE |
| 34 | 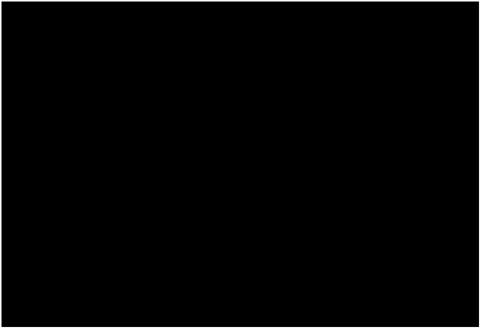 | 2-{3-[(4-METHYL-CYCLOHEXANECARBONYL)-(1-METH- YL-PIPERIDIN-4-YL)-AMINO]-5-PHENYL-THIO- PHENE-2-CARBONYLOXYMETHOXYCARBONYL}-PY- RROLIDINIUM; BIS-TRIFLUORO-ACETATE |
| 35 | 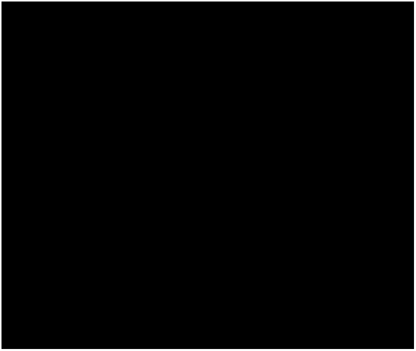 | 4-[[2-(1-ISOBUTYRYLOXY-ETHOXYCARBONYL)-5-PHE- NYL-THIOPHEN-3-YL]-(4-METHYL-CYCLO- HEXANECARBONYL)-AMINO]-1-METHYL-PIPE- RIDINIUM; CHLORIDE |
| 36 | 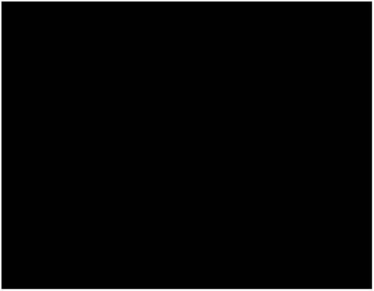 | 3-[(4-METHYL-CYCLOHEXANECARBONYL)-(1-METH- YL-PIPERIDIN-4-YL)-AMINO]-5-PHENYL-THIO- PHENE-2-CARBOXYLIC ACID PYRIDIN-2-YL ESTER |
| 37 | 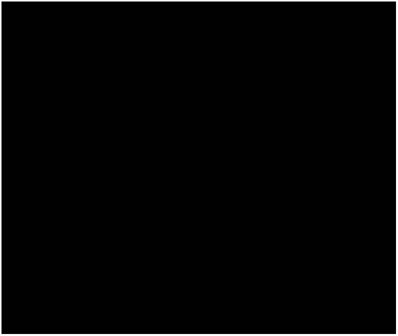 | 4-[[2-(1-ACETOXY-1-METHYL-ETHOXYCARBONYL)-5-PHE- NYL-THIOPHEN-3-YL]-(4-METHYL-CYCLO- HEXANECARBONYL)-AMINO]-1-METHYL-PIPE- RIDINIUM; CHLORIDE |
| 38 | 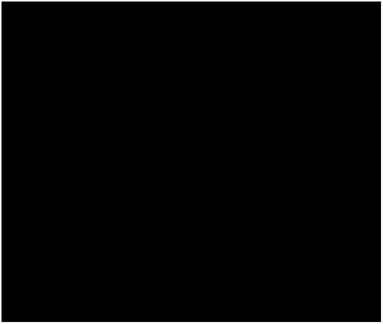 | 3-[(4-METHYL-CYCLOHEXANECARBONYL)-(1-METH- YL-PIPERIDIN-4-YL)-AMINO]-5-PHENYL-THIO- PHENE-2-CARBOXYLIC ACID 2-OXO-PYR- ROLIDIN-1-YLMETHYL ESTER |
| 39 | 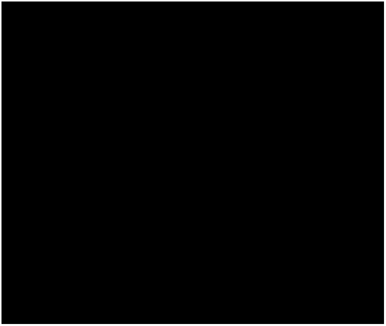 | 1-METHYL-4-[(4-METHYL-CYCLOHEXANECARBONYL)-(2-PHE- NOXYCARBONYL-5-PHENYL-THIOPHEN-3-YL)-A- MINO]-PIPERIDINIUM; CHLORIDE |
| 40 | 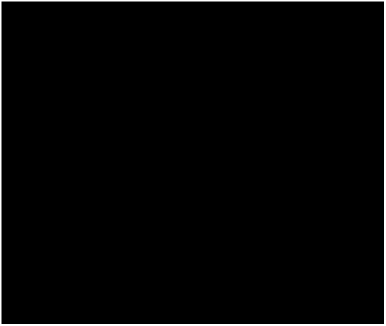 | 1-METHYL-4-{(4-METHYL-CYCLOHEXANECARBONYL)-[5-PHE- NYL-2-(PYRIDIN-3-YLOXY- CARBONYL)-THIOPHEN-3-YL]-AMINO}-PIPE- RIDINIUM; CHLORIDE |
| 41 | 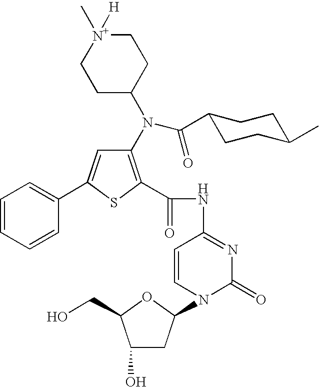 | 4-[{2-[1-(4-HYDROXY-5-HYDROXYMETHYL-TETRA- HYDRO-FURAN-2-YL)-2-OXO-1,2-DIHYDRO-PY- RIMIDIN-4-YLCARBAMOYL]-5-PHENYL-THIOPHEN-3-YL}-(4-METH- YL-CYCLOHEXANECARBONYL)-AMINO}-1-METH- YL-PIPERIDINIUM CHLORIDE |
| 42 | 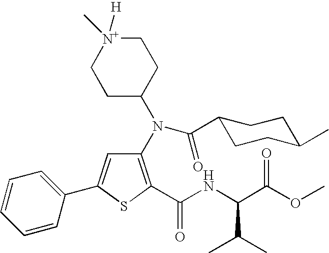 | 4-[[2-(1-METHOXYCARBONYL-2-METHYL-PRO- PYLCARBAMOYL)-5-PHENYL-THIOPHEN-3-YL]-(4-METH- YL-CYCLOHEXANECARBONYL)-AMINO]-1-METH- YL-PIPERIDINIUM CHLORIDE |
| 43 | 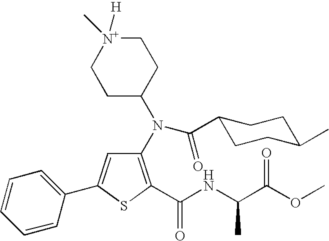 | 4-[[2-(1-METHOXYCARBONYL-ETHYLCARBAMOYL)-5-PHE- NYL-THIOPHEN-3-YL]-(4-METHYL-CYCLO- HEXANECARBONYL)-AMINO]-1-METHYL-PIPE- RIDINIUM |

### EXAMPLE 6

### Evaluation of Bioavailability in Male CD-1 Mice

Male CD-1 mice were dosed with the compound by iv (tail injection; vehicule: 45% β-cyclodextrin in saline) and by po (gavage; vehicule: 0.5% carboxy Methyl Cellulose+5% tween 80+saline).

Parent compound was administered at a dose of 20 mg/kg (i.v.) and 130 mg/kg (p.o.) whereas compound #1 and compound #2 were administered at 25 mg/kg (i.v. and p.o.)

The blood was collected in EDTA tube. Time points collected for iv are: 2, 5, 15, 30, 45, 60, 90, 120, 180 and 240 minutes post injection and for po are: 5, 15, 30, 45, 60, 90, 120, 180 and 240 minutes post injection. Three mice per time point. The blood was centrifuged to obtain plasma samples.

Plasma samples were extracted by protein precipitation with acetonitrile, evaporated and reconstituted with 50% MeOH/H₂O. 30 μL was injected for the parent compound and for compound #1 and compound #2 10 μL was injected.

The standard curve range was between 0.1 to 30 μg/ml for the parent compound, 0.5 to 5 μg/ml for compound #1 and 0.01 to 2 μg/ml for compound #2. Adequate QC were performed.

The analysis was performed with a HPLC Agilent 1100 equipped with a MSD mass spectrometry detector with API-ESI source (LC/MS) supported by ChemStation software.

Analytical condition: The column was a Luna C18 (2) 5 μm 2×50 mm distributed by Phenomenex. A linear gradient of acetonitrile in 10 mM ammonium formate pH 6 was done at a flow rate of 0.25 ml/min.

### EXAMPLE 9

### Bioavailability of Selected Compounds

| Compound # | Bioavailability* |
|---|---|
| Parent | 0.4% |
| compound | |
| compound #1 | 8.7% |
| compound #2 | 3.8% |

*Evaluated by the method described in Example 8

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

What is claimed:

1. A compound represented by formula I: 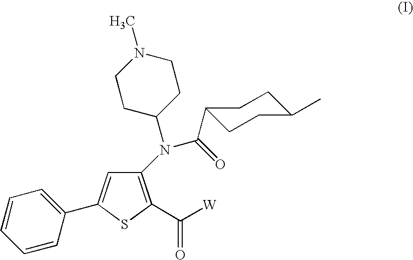
or pharmaceutically acceptable salts thereof;
wherein;
W is C₁₋₂ alkyloxy, C₆₋₁₂ arylalkyloxy, amino acid ester, nucleoside, C₆₋₁₂ heteroaralkyloxy, C₆ aryloxy, 5-6 membered heteroaryloxy, 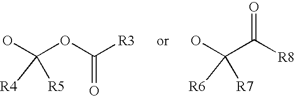
R₃ and R₈ are each independently chosen from C₁₋₁₂ alkyl, C₆₋₁₀ aryl, C₆₋₁₀ arylalkyl, C₃₋₁₀ heterocycle, C₃₋₁₂ heteroaralkyl, C₆₋₁₂ aralkyl, C₁₋₁₂ alkyloxy, C₆₋₁₀ aryloxy or NR₁₀R₁₁ wherein
R₁₀ and R₁₁ are each independently chosen from H, C₁₋₁₂ alkyl, C₆₋₁₂ aryl, C₃₋₁₀ heterocycle, C₆₋₁₂ aralkyl or C₃₋₁₀ heteroaralkyl.
R₄ and R₅ are each independently chosen from H, C₁₋₁₂ alkyl, C₆₋₁₀ aryl, —O(CO)C₁₋₆ alkyl or C₃₋₁₀ heterocycle;
R₆ and R₇ are each independently chosen from H, C₁₋₁₂ alkyl, C₆₋₁₀ aryl, —O(CO)C₁₋₆ alkyl or C₃₋₁₀ heterocycle.

2. A compound of claim 1, wherein W is C₁₋₁₂ alkyloxy, amino acid ester,

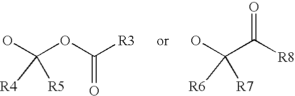

3. A compound of claim 1, wherein, R₃ and R₈ are each independently chosen from C₁₋₁₂ alkyl, C₆₋₁₀ aryl, C₆₋₁₀ arylalkyl, C₃₋₁₀ heterocycle, C₃₋₁₂ heteroaralkyl, C₆₋₁₂ aralkyl, C₁₋₁₂ alkyloxy, C₆₋₁₀ aryloxy or NR₁₀R₁₁ wherein R₁₀ and R₁₁ are each independently chosen from H, C₁₋₁₂ alkyl, C₆₋₁₂ aryl, C₃₋₁₀ heterocycle, C₆₋₁₂ aralkyl or C₃₋₁₀ heteroaralkyl.

4. A compound of claim 1, wherein R₄ and R₅ are each independently chosen from H, C₁₋₁₂ alkyl, C₆₋₁₀ aryl, —O(CO)C₁₋₆ alkyl or C₃₋₁₀ heterocycle.

5. A compound of claim 1, wherein R4 and R5 are taken together to form a 3-6 membered cycloalkyl.

6. A compound of claim 1, wherein R₆ and R₇ are each independently chosen from H, C₁₋₁₂ alkyl, C₆₋₁₀ aryl, —O(CO)C₁₋₆ alkyl or C₃₋₁₀ heterocycle;

7. A compound of claim 1, wherein R₆ and R₇ are taken together to form a 3-6 membered cycloalkyl.

8. A compound of claim 1, wherein R₁₀ and R₁₁ are each independently chosen from H, C₁₋₁₂ alkyl, C₃₋₁₀ heterocycle.

9. A compound selected from:
3-[(4-METHYL-CYCLOHEXANECARBONYL)-(1-METHYL-PIPERIDIN-4-YL)-AMINO]-5-PHENYL-THIOPHENE-2-CARBOXYLIC ACID 2,2-DIMETHYL-PROPIONYLOXYMETHY;
4-[(2-ISOPROPOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM; CHLORIDE
4-[(2-ISOPROPYLCARBAMOYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
1-METHYL-4-{(4-METHYL-CYCLOHEXANECARBONYL)-[2-(5-METHYL-2-OXO-[1,3]DIOXOL-4-YLMETHOXYCARBONYL)-5-PHENYL-THIOPHEN-3-YL]-AMINO}-PIPERIDINIUM;
4-[[2-(1-ISOPROPOXYCARBONYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
4-[{2-[1-(2,2-DIMETHYL-PROPIONYLOXY)-ETHOXYCARBONYL]-5-PHENYL-THIOPHEN-3-YL}-(4-METHYL-CYCLOHEXANECARBONYL)-AMINO]-1-METHYL-PIPERIDINIUM CHLORIDE;
4-[[2-(ISOPROPOXYCARBONYLOXY-PHENYL-METHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
4-[(2-CYCLOHEXYLOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
4-[{2-[(2,2-DIMETHYL-PROPIONYLOXY)-PHENYL-METHOXYCARBONYL]-5-PHENYL-THIOPHEN-3-YL}-(4-METHYL-CYCLOHEXANECARBONYL)-AMINO]-1-METHYL-PIPERIDINIUM CHLORIDE;
1-METHYL-4-[(4-METHYL-CYCLOHEXANECARBONYL)-(5-PHENYL-2-PROPIONYLOXYMETHOXYCARBONYL-THIOPHEN-3-YL)-AMINO]-PIPERIDINIUM; CHLORIDE;
4-[[2-(FURAN-2-CARBONYLOXYMETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
4-[(2-BENZOYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
4-[(2-CYCLOHEXANECARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
1-METHYL-4-[(4-METHYL-CYCLOHEXANECARBONYL)-(5-PHENYL-2-SUCCINYL-17(3-TERT-BUTOXYCARBONYLMETHYL-CARBAMOYL)-METHYL-PROPYL)-7,12-DIHYDROXY-10,13-DIMETHYL-HEXADECAHYDRO-CYCLOPENTA(A)PHENANTHREN-3-YLOYMETHOXYCARBONYL-THIOPHEN-3-YL)AMINO-PIPERIDINIUM CHLORIDE;
METHYL-4-[(4-METHYL-CYCLOHEXANECARBONYL)-(5-PHENYL-2-SUCCINYL-17(3-CARBONYLMETHYL-CARBAMOYL)-METHYL-PROPYL)-7,12-DIHYDROXY-10,13-DIMETHYL-HEXADECAHYDRO-CYCLOPENTA(A)PHENANTHREN-3-YLOYMETHOXYCARBONYL-THIOPHEN-3-YL)AMINO-PIPERIDINIUM CHLORIDE;
4-[(2-ETHOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
1-METHYL-4-[(4-METHYL-CYCLOHEXANECARBONYL)-(2-PHENOXYCARBONYLOXYMETHOXYCARBONYL-5-PHENYL-THIOPHEN-3-YL)-AMINO]-PIPERIDINIUM CHLORIDE;
1-METHYL-4-{(4-METHYL-CYCLOHEXANECARBONYL)-[2-(MORPHOLINE-4-CARBONYLOXYMETHOXYCARBONYL)-5-PHENYL-THIOPHEN-3-YL]-AMINO}-PIPERIDINIUM CHLORIDE;
4-[(2-[1-(2,2-DIMETHYL-PROPIONYLOXY)-2-METHYL-PROPOXYCARBONYL]-5-PHENYL-THIOPHEN-3-YL}-(4-METHYL-CYCLOHEXANECARBONYL)-AMINO]-1-METHYL-PIPERIDINIUM CHLORIDE;
4-[(2-SEC-BUTOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
4-[[2-(1-ISOPROPOXYCARBONYLOXY-2-METHYL-PROPOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
4-[(2-SEC-BUTOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM; CHLORIDE;
3-[(4-METHYL-CYCLOHEXANECARBONYL)-(1-METHYL-PIPERIDIN-4-YL)-AMINO]-5-PHENYL-THIOPHENE-2-CARBOXYLIC ACID TERT-BUTOXYCARBONYLAMINOACETOXYMETHYL ESTER;
3-[(4-METHYL-CYCLOHEXANECARBONYL)-(1-METHYL-PIPERIDIN-4-YL)-AMINO]-5-PHENYL-THIOPHENE-2-CARBOXYLIC ACID 2-TERT-BUTOXYCARBONYLAMINO-3-METHYL-BUTYRYLOXYMETHYL ESTER;
3-[(4-METHYL-CYCLOHEXANECARBONYL)-(1-METHYL-PIPERIDIN-4-YL)-AMINO]-5-PHENYL-THIOPHENE-2-CARBOXYLIC ACID AMINOACETOXYMETHYL ESTER, BIS TRIFLUOROACETATE SALT;
3-[(4-METHYL-CYCLOHEXANECARBONYL)-(1-METHYL-PIPERIDIN-4-YL)-AMINO]-5-PHENYL-THIOPHENE-2-CARBOXYLIC ACID 2-AMINO-3-METHYL-BUTYRYLOXYMETHYL ESTER, BIS TRIFLUOROACETATE SALT;
4-[[2-(1-ISOPROPOXYCARBONYLOXY-1-METHYL-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
3-[(4-METHYL-CYCLOHEXANECARBONYL)-(1-METHYL-PIPERIDIN-4-YL)-AMINO]-5-PHENYL-THIOPHENE-2-CARBOXYLIC ACID 1-(1-METHYL-CYCLOHEXANECARBONYLOXY)-ETHYL ESTER;
4-[[2-(1-TERT-BUTOXYCARBONYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
1-METHYL-4-((4-METHYL-CYCLOHEXANECARBONYL)-{2-[1-(1-CYCLOHEXANECARBONYLOXY)-ETHOXYCARBONYL]-5-PHENYL-THIOPHEN-3-YL}-AMINO)-PIPERIDINIUM;
PYRROLIDINE-1,2-DICARBOXYLIC ACID 1-TERT-BUTYL ESTER 2-{3-[(4-METHYL-CYCLOHEXANECARBONYL)-(1-METHYL-PIPERIDIN-4-YL)-AMINO]-5-PHENYL-THIOPHENE-2-CARBONYLOXYMETHYL} ESTER;
4-Methyl-piperazine-1-carboxylic acid 3-[(4-methyl-cyclohexanecarbonyl)-(1-methyl-piperidin-4-yl)-amino]-5-phenyl-thiophene-2-carbonyloxymethyl ester dihydrochloride salt;
4-[[2-(1-CYCLOHEXYLOXYCARBONYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM; CHLORIDE;
2-{3-[(4-METHYL-CYCLOHEXANECARBONYL)-(1-METHYL-PIPERIDIN-4-YL)-AMINO]-5-PHENYL-THIOPHENE-2-CARBONYLOXYMETHOXYCARBONYL}-PYRROLIDINIUM; BIS-TRIFLUORO-ACETATE;
4-[[2-(1-ISOBUTYRYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM; CHLORIDE;
3-[(4-METHYL-CYCLOHEXANECARBONYL)-(1-METHYL-PIPERIDIN-4-YL)-AMINO]-5-PHENYL-THIOPHENE-2-CARBOXYLIC ACID PYRIDIN-2-YL ESTER;
4-[[2-(1-ACETOXY-1-METHYL-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
3-[(4-METHYL-CYCLOHEXANECARBONYL)-(1-METHYL-PIPERIDIN-4-YL)-AMINO]-5-PHENYL-THIOPHENE-2-CARBOXYLIC ACID 2-OXO-PYRROLIDIN-1-YLMETHYL ESTER;
1-METHYL-4-[(4-METHYL-CYCLOHEXANECARBONYL)-(2-PHENOXYCARBONYL-5-PHENYL-THIOPHEN-3-YL)-AMINO]-PIPERIDINIUM CHLORIDE;
1-METHYL-4-{(4-METHYL-CYCLOHEXANECARBONYL)-[5-PHENYL-2-(PYRIDIN-3-YLOXYCARBONYL)-THIOPHEN-3-YL]-AMINO}-PIPERIDINIUM; CHLORIDE;
4-[{2-[1-(4-HYDROXY-5-HYDROXYMETHYL-TETRAHYDRO-FURAN-2-YL)-2-OXO-1,2-DIHYDRO-PYRIMIDIN-4-YLCARBAMOYL]-5-PHENYL-THIOPHEN-3-YL}-(4-METHYL-CYCLOHEXANECARBONYL)-AMINO]-1-METHYL-PIPERIDINIUM CHLORIDE;
4-[[2-(1-METHOXYCARBONYL-2-METHYL-PROPYLCARBAMOYL)-5-PHENYL-THIOPHEN-3-YL]-(4-METHYL-CYCLOHEXANECARBONYL)-AMINO]-1-METHYL-PIPERIDINIUM CHLORIDE;
4-[[2-(1-METHOXYCARBONYL-ETHYLCARBAMOYL)-5-PHENYL-THIOPHEN-3-YL]-(4-METHYL-CYCLOHEXANECARBONYL)-AMINO]-1-METHYL-PIPERIDINIUM;
or pharmaceutically acceptable salts thereof.

10. A method for treating or preventing a Flaviviridae viral infection in a host comprising administering to the host a therapeutically effective amount of at least one compound of formula I: 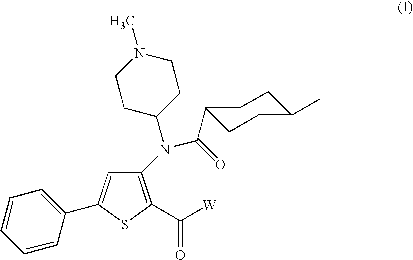
or pharmaceutically acceptable salts thereof;
wherein;
W is C₁₋₁₂ alkyloxy, C₆₋₁₂ arylalkyloxy, amino acid ester, nucleoside, C₆₋₁₂ heteroaralkyloxy, C₆ aryloxy, 5-6 membered heteroaryloxy, 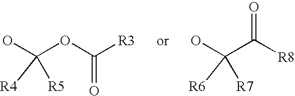
R₃ and R₈ are each independently chosen from C₁₋₁₂ alkyl, C₆₋₁₀ aryl, C₆₋₁₀ arylalkyl, C₃₋₁₀ heterocycle, C₃₋₁₂ heteroaralkyl, C₆₋₁₂ aralkyl, C₁₋₁₂ alkyloxy, C₆₋₁₀ aryloxy or NR₁₀R₁₁ wherein
R₁₀ and R₁₁ are each independently chosen from H, C₁₋₁₂ alkyl, C₆₋₁₂ aryl, C₃₋₁₀ heterocycle, C₆₋₁₂ aralkyl or C₃₋₁₀ heteroaralkyl;
R₄ and R₅ are each independently chosen from H, C₁₋₁₂ alkyl, C₆₋₁₀ aryl, —O(CO)C₁₋₆ alkyl or C₃₋₁₀ heterocycle;
R₆ and R₇ are each independently chosen from H, C₁₋₁₂ alkyl, C₆₋₁₀ aryl, —O(CO)C₁₋₆ alkyl or C₃₋₁₀ heterocycle.

11. A method of claim 10, further comprising administering at least one additional agent chosen from viral serine protease inhibitor, viral polymerase inhibitor, viral helicase inhibitor, immunomudulating agent, antioxydant agent, antibacterial agent, therapeutic vaccine, hepatoprotectant agent or antisense agent.

12. A method of claim 11, wherein the additional agent is interferon α, ribavirin, silybum marianum, interleukine-12, amantadine, ribozyme, thymosin, N-acetyl cysteine or cyclosporin.

13. A method according to any of claims 10 to 12, wherein the Flaviviridea viral infection is hepatitis C viral infection (HCV).

14. A method for treating or preventing a Flaviviridae viral infection in a host comprising administering to the host a therapeutically effective amount of at least one compound selected from:
3-[(4-METHYL-CYCLOHEXANECARBONYL)-(1-METHYL-PIPERIDIN-4-YL)-AMINO]-5-PHENYL-THIOPHENE-2-CARBOXYLIC ACID 2,2-DIMETHYL-PROPIONYLOXYMETHY;
4-[(2-ISOPROPOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM; CHLORIDE
4-[(2-ISOPROPYLCARBAMOYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
1-METHYL-4-{(4-METHYL-CYCLOHEXANECARBONYL)-[2-(5-METHYL-2-OXO-[1,3]DIOXOL-4-YLMETHOXYCARBONYL)-5-PHENYL-THIOPHEN-3-YL]-AMINO}-PIPERIDINIUM;
4-[[2-(1-ISOPROPOXYCARBONYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
4-[{2-[1-(2,2-DIMETHYL-PROPIONYLOXY)-ETHOXYCARBONYL]-5-PHENYL-THIOPHEN-3-YL}-(4-METHYL-CYCLOHEXANECARBONYL)-AMINO]-1-METHYL-PIPERIDINIUM CHLORIDE;
4-[[2-(ISOPROPOXYCARBONYLOXY-PHENYL-METHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
4-[(2-CYCLOHEXYLOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
4-[{2-[(2,2-DIMETHYL-PROPIONYLOXY)-PHENYL-METHOXYCARBONYL]-5-PHENYL-THIOPHEN-3-YL}-(4-METHYL-CYCLOHEXANECARBONYL)-AMINO]-1-METHYL-PIPERIDINIUM CHLORIDE;
1-METHYL-4-[(4-METHYL-CYCLOHEXANECARBONYL)-(5-PHENYL-2-PROPIONYLOXYMETHOXYCARBONYL-THIOPHEN-3-YL)-AMINO]-PIPERIDINIUM; CHLORIDE;
4-[[2-(FURAN-2-CARBONYLOXYMETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
4-[(2-BENZOYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
4-[(2-CYCLOHEXANECARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
1-METHYL-4-[(4-METHYL-CYCLOHEXANECARBONYL)-(5-PHENYL-2-SUCCINYL-17(3-TERT-BUTOXYCARBONYLMETHYL-CARBAMOYL)-METHYL-PROPYL)-7,12-DIHYDROXY-10,13-DIMETHYL-HEXADECAHYDRO-CYCLOPENTA(A)PHENANTHREN-3-YLOYMETHOXYCARBONYL-THIOPHEN-3-YL)AMINO-PIPERIDINIUM CHLORIDE;
METHYL-4-[(4-METHYL-CYCLOHEXANECARBONYL)-(5-PHENYL-2-SUCCINYL-17(3-CARBONYLMETHYL-CARBAMOYL)-METHYL-PROPYL)-7,12-DIHYDROXY-10,13-DIMETHYL-HEXADECAHYDRO-CYCLOPENTA(A)PHENANTHREN-3-YLOYMETHOXYCARBONYL-THIOPHEN-3-YL)AMINO-PIPERIDINIUM CHLORIDE;
4-[(2-ETHOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
1-METHYL-4-[(4-METHYL-CYCLOHEXANECARBONYL)-(2-PHENOXYCARBONYLOXYMETHOXYCARBONYL-5-PHENYL-THIOPHEN-3-YL)-AMINO]-PIPERIDINIUM CHLORIDE;
1-METHYL-4-{(4-METHYL-CYCLOHEXANECARBONYL)-[2-(MORPHOLINE-4-CARBONYLOXYMETHOXYCARBONYL)-5-PHENYL-THIOPHEN-3-YL]-AMINO}-PIPERIDINIUM CHLORIDE;
4-[{2-[1-(2,2-DIMETHYL-PROPIONYLOXY)-2-METHYL-PROPOXYCARBONYL]-5-PHENYL-THIOPHEN-3-YL}-(4-METHYL-CYCLOHEXANECARBONYL)-AMINO]-1-METHYL-PIPERIDINIUM CHLORIDE;
4-[(2-SEC-BUTOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
4-[[2-(1-ISOPROPOXYCARBONYLOXY-2-METHYL-PROPOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
4-[(2-SEC-BUTOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM; CHLORIDE;
3-[(4-METHYL-CYCLOHEXANECARBONYL)-(1-METHYL-PIPERIDIN-4-YL)-AMINO]-5-PHENYL-THIOPHENE-2-CARBOXYLIC ACID TERT-BUTOXYCARBONYLAMINOACETOXYMETHYL ESTER;
3-[(4-METHYL-CYCLOHEXANECARBONYL)-(1-METHYL-PIPERIDIN-4-YL)-AMINO]-5-PHENYL-THIOPHENE-2-CARBOXYLIC ACID 2-TERT-BUTOXYCARBONYLAMINO-3-METHYL-BUTYRYLOXYMETHYL ESTER;
3-[(4-METHYL-CYCLOHEXANECARBONYL)-(1-METHYL-PIPERIDIN-4-YL)-AMINO]-5-PHENYL-THIOPHENE-2-CARBOXYLIC ACID AMINOACETOXYMETHYL ESTER, BIS TRIFLUOROACETATE SALT;
3-[(4-METHYL-CYCLOHEXANECARBONYL)-(1-METHYL-PIPERIDIN-4-YL)-AMINO]-5-PHENYL-THIOPHENE-2-CARBOXYLIC ACID 2-AMINO-3-METHYL-BUTYRYLOXYMETHYL ESTER, BIS TRIFLUOROACETATE SALT;
4-[[2-(1-ISOPROPOXYCARBONYLOXY-1-METHYL-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
3-[(4-METHYL-CYCLOHEXANECARBONYL)-(1-METHYL-PIPERIDIN-4-YL)-AMINO]-5-PHENYL-THIOPHENE-2-CARBOXYLIC ACID 1-(1-METHYL-CYCLOHEXANECARBONYLOXY)-ETHYL ESTER;
4-[[2-(1-TERT-BUTOXYCARBONYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
1-METHYL-4-((4-METHYL-CYCLOHEXANECARBONYL)-{2-[1-(1-METHYL-CYCLOHEXANECARBONYLOXY)-ETHOXYCARBONYL]-5-PHENYL-THIOPHEN-3-YL}-AMINO)-PIPERIDINIUM;
PYRROLIDINE-1,2-DICARBOXYLIC ACID 1-TERT-BUTYL ESTER 2-{3-[(4-METHYL-CYCLOHEXANECARBONYL)-(1-METHYL-PIPERIDIN-4-YL)-AMINO]-5-PHENYL-THIOPHENE-2-CARBONYLOXYMETHYL} ESTER;
4-Methyl-piperazine-1-carboxylic acid 3-[(4-methyl-cyclohexanecarbonyl)-(1-methyl-piperidin-4-yl)-amino]-5-phenyl-thiophene-2-carbonyloxymethyl ester dihydrochloride salt;
4-[[2-(1-CYCLOHEXYLOXYCARBONYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM; CHLORIDE;
2-{3-[(4-METHYL-CYCLOHEXANECARBONYL)-(1-METHYL-PIPERIDIN-4-YL)-AMINO]-5-PHENYL-THIOPHENE-2-CARBONYLOXYMETHOXYCARBONYL}-PYRROLIDINIUM; BIS-TRIFLUORO-ACETATE;
4-[[2-(1-ISOBUTYRYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM; CHLORIDE;
3-[(4-METHYL-CYCLOHEXANECARBONYL)-(1-METHYL-PIPERIDIN-4-YL)-AMINO]-5-PHENYL-THIOPHENE-2-CARBOXYLIC ACID PYRIDIN-2-YL ESTER;
4-[[2-(1-ACETOXY-1-METHYL-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
3-[(4-METHYL-CYCLOHEXANECARBONYL)-(1-METHYL-PIPERIDIN-4-YL)-AMINO]-5-PHENYL-THIOPHENE-2-CARBOXYLIC ACID 2-OXO-PYRROLIDIN-1-YLMETHYL ESTER;
1-METHYL-4-[(4-METHYL-CYCLOHEXANECARBONYL)-(2-PHENOXYCARBONYL-5-PHENYL-THIOPHEN-3-YL)-AMINO]-PIPERIDINIUM CHLORIDE;
1-METHYL-4-{(4-METHYL-CYCLOHEXANECARBONYL)-[5-PHENYL-2-(PYRIDIN-3-YLOXYCARBONYL)-THIOPHEN-3-YL]-AMINO}-PIPERIDINIUM; CHLORIDE;
4-[{2-[1-(4-HYDROXY-5-HYDROXYMETHYL-TETRAHYDRO-FURAN-2-YL)-2-OXO-1,2-DIHYDRO-PYRIMIDIN-4-YLCARBAMOYL]-5-PHENYL-THIOPHEN-3-YL}-(4-METHYL-CYCLOHEXANECARBONYL)-AMINO]-1-METHYL-PIPERIDINIUM CHLORIDE;
4-[[2-(1-METHOXYCARBONYL-2-METHYL-PROPYLCARBAMOYL)-5-PHENYL-THIOPHEN-3-YL]-(4-METHYL-CYCLOHEXANECARBONYL)-AMINO]-1-METHYL-PIPERIDINIUM CHLORIDE;
4-[[2-(1-METHOXYCARBONYL-ETHYLCARBAMOYL)-5-PHENYL-THIOPHEN-3-YL]-(4-METHYL-CYCLOHEXANECARBONYL)-AMINO]-1-METHYL-PIPERIDINIUM;
or pharmaceutically acceptable salts thereof.

15. A pharmaceutical composition comprising at least one compound of formula I: 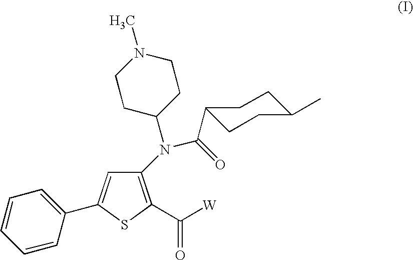
or pharmaceutically acceptable salts thereof;
wherein;
W is C₁₋₁₂ alkyloxy, C₆₋₁₂ arylalkyloxy, amino acid ester, nucleoside, C₆₋₁₂ heteroaralkyloxy, C₆ aryloxy, 5-6 membered heteroaryloxy, 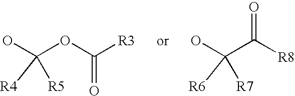
R₃ and R₈ are each independently chosen from C₁₋₁₂ alkyl, C₆₋₁₀ aryl, C₆₋₁₀ arylalkyl, C₃₋₁₀ heterocycle, C₃₋₁₂ heteroaralkyl, C₆₋₁₂ aralkyl, C₁₋₁₂ alkyloxy, C₆₋₁₀ aryloxy or NR₁₀R₁₁ wherein
R₁₀ and R₁₁ are each independently chosen from H, C₁₋₁₂ alkyl, C₆₋₁₂ aryl, C₃₋₁₀ heterocycle, C₆₋₁₂ aralkyl or C₃₋₁₀ heteroaralkyl;
R₄ and R₅ are each independently chosen from H, C₁₋₁₂ alkyl, C₆₋₁₀ aryl, —O(CO)C₁₋₆ alkyl or C₃₋₁₀ heterocycle;
R₆ and R7 are each independently chosen from H, C₁₋₁₂ alkyl, C₆₋₁₀ aryl, —O(CO)C₁₋₆ alkyl or C₃₋₁₀ heterocycle;
and at least one pharmaceutically acceptable carrier or excipient.

16. A pharmaceutical composition according to claim 15, further comprising at least one additional agent chosen from viral serine protease inhibitor, viral polymerase inhibitor, viral helicase inhibitor, immunomudulating agent, antioxydant agent, antibacterial agent, therapeutic vaccine, hepatoprotectant agent or antisense agent.

17. A pharmaceutical composition of claim 16, wherein the additional agent is interferon α, ribavirin, silybum marianum, interleukine-12, amantadine, ribozyme, thymosin, N-acetyl cysteine or cyclosporin.

18. A pharmaceutical composition comprising at least one compound selected from:
3-[(4-METHYL-CYCLOHEXANECARBONYL)-(1-METHYL-PIPERIDIN-4-YL)-AMINO]-5-PHENYL-THIOPHENE-2-CARBOXYLIC ACID 2,2-DIMETHYL-PROPIONYLOXYMETHY;
4-[(2-ISOPROPOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM; CHLORIDE
4-[(2-ISOPROPYLCARBAMOYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
1-METHYL-4-{(4-METHYL-CYCLOHEXANECARBONYL)-[2-(5-METHYL-2-OXO-[1,3]DIOXOL-4-YLMETHOXYCARBONYL)-5-PHENYL-THIOPHEN-3-YL]-AMINO}-PIPERIDINIUM;
4-[[2-(1-ISOPROPOXYCARBONYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
4-[{2-[1-(2,2-DIMETHYL-PROPIONYLOXY)-ETHOXYCARBONYL]-5-PHENYL-THIOPHEN-3-YL}-(4-METHYL-CYCLOHEXANECARBONYL)-AMINO]-1-METHYL-PIPERIDINIUM CHLORIDE;
4-[[2-(ISOPROPOXYCARBONYLOXY-PHENYL-METHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
4-[(2-CYCLOHEXYLOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
4-[{2-[(2,2-DIMETHYL-PROPIONYLOXY)-PHENYL-METHOXYCARBONYL]-5-PHENYL-THIOPHEN-3-YL}-(4-METHYL-CYCLOHEXANECARBONYL)-AMINO]-1-METHYL-PIPERIDINIUM CHLORIDE;
1-METHYL-4-[(4-METHYL-CYCLOHEXANECARBONYL)-(5-PHENYL-2-PROPIONYLOXYMETHOXYCARBONYL-THIOPHEN-3-YL)-AMINO]-PIPERIDINIUM; CHLORIDE;
4-[[2-(FURAN-2-CARBONYLOXYMETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
4-[(2-BENZOYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
4-[(2-CYCLOHEXANECARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
1-METHYL-4-[(4-METHYL-CYCLOHEXANECARBONYL)-(5-PHENYL-2-SUCCINYL-17(3-TERT-BUTOXYCARBONYLMETHYL-CARBAMOYL)-METHYL-PROPYL)-7,12-DIHYDROXY-10,13-DIMETHYL-HEXADECAHYDRO-CYCLOPENTA(A)PHENANTHREN-3-YLOYMETHOXYCARBONYL-THIOPHEN-3-YL)AMINO-PIPERIDINIUM CHLORIDE;
METHYL-4-[(4-METHYL-CYCLOHEXANECARBONYL)-(5-PHENYL-2-SUCCINYL-17(3-CARBONYLMETHYL-CARBAMOYL)-METHYL-PROPYL)-7,12-DIHYDROXY-10,13-DIMETHYL-HEXADECAHYDRO-CYCLOPENTA(A)PHENANTHREN-3-YLOYMETHOXYCARBONYL-THIOPHEN-3-YL)AMINO-PIPERIDINIUM CHLORIDE;
4-[(2-ETHOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
1-METHYL-4-[(4-METHYL-CYCLOHEXANECARBONYL)-(2-PHENOXYCARBONYLOXYMETHOXYCARBONYL-5-PHENYL-THIOPHEN-3-YL)-AMINO]-PIPERIDINIUM CHLORIDE;
1-METHYL-4-{(4-METHYL-CYCLOHEXANECARBONYL)-[2-(MORPHOLINE-4-CARBONYLOXYMETHOXYCARBONYL)-5-PHENYL-THIOPHEN-3-YL]-AMINO}-PIPERIDINIUM CHLORIDE;
4-[{2-[1-(2,2-DIMETHYL-PROPIONYLOXY)-2-METHYL-PROPOXYCARBONYL]-5-PHENYL-THIOPHEN-3-YL}-(4-METHYL-CYCLOHEXANECARBONYL)-AMINO]-1-METHYL-PIPERIDINIUM CHLORIDE;
4-[(2-SEC-BUTOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
4-[[2-(1-ISOPROPOXYCARBONYLOXY-2-METHYL-PROPOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
4-[(2-SEC-BUTOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM; CHLORIDE;
3-[(4-METHYL-CYCLOHEXANECARBONYL)-(1-METHYL-PIPERIDIN-4-YL)-AMINO]-5-PHENYL-THIOPHENE-2-CARBOXYLIC ACID TERT-BUTOXYCARBONYLAMINOACETOXYMETHYL ESTER;
3-[(4-METHYL-CYCLOHEXANECARBONYL)-(1-METHYL-PIPERIDIN-4-YL)-AMINO]-5-PHENYL-THIOPHENE-2-CARBOXYLIC ACID 2-TERT-BUTOXYCARBONYLAMINO-3-METHYL-BUTYRYLOXYMETHYL ESTER;
3-[(4-METHYL-CYCLOHEXANECARBONYL)-(1-METHYL-PIPERIDIN-4-YL)-AMINO]-5-PHENYL-THIOPHENE-2-CARBOXYLIC ACID AMINOACETOXYMETHYL ESTER, BIS TRIFLUOROACETATE SALT;
3-[(4-METHYL-CYCLOHEXANECARBONYL)-(1-METHYL-PIPERIDIN-4-YL)-AMINO]-5-PHENYL-THIOPHENE-2-CARBOXYLIC ACID 2-AMINO-3-METHYL-BUTYRYLOXYMETHYL ESTER, BIS TRIFLUOROACETATE SALT;
4-[[2-(1-ISOPROPOXYCARBONYLOXY-1-METHYL-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
3-[(4-METHYL-CYCLOHEXANECARBONYL)-(1-METHYL-PIPERIDIN-4-YL)-AMINO]-5-PHENYL-THIOPHENE-2-CARBOXYLIC ACID 1-(1-METHYL-CYCLOHEXANECARBONYLOXY)-ETHYL ESTER;
4-[[2-(1-TERT-BUTOXYCARBONYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
1-METHYL-4-((4-METHYL-CYCLOHEXANECARBONYL)-{2-[1-(1-METHYL-CYCLOHEXANECARBONYLOXY)-ETHOXYCARBONYL-]-5-PHENYL-THIOPHEN-3-YL}-AMINO)-PIPERIDINIUM;
PYRROLIDINE-1,2-DICARBOXYLIC ACID 1-TERT-BUTYL ESTER 2-{3-[(4-METHYL-CYCLOHEXANECARBONYL)-(1-METHYL-PIPERIDIN-4-YL)-AMINO]-5-PHENYL-THIOPHENE-2-CARBONYLOXYMETHYL} ESTER;
4-Methyl-piperazine-1-carboxylic acid 3-[(4-methyl-cyclohexanecarbonyl)-(1-methyl-piperidin-4-yl)-amino]-5-phenyl-thiophene-2-carbonyloxymethyl ester dihydrochloride salt;
4-[[2-(1-CYCLOHEXYLOXYCARBONYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM; CHLORIDE;
2-{3-[(4-METHYL-CYCLOHEXANECARBONYL)-(1-METHYL-PIPERIDIN-4-YL)-AMINO]-5-PHENYL-THIOPHENE-2-CARBONYLOXYMETHOXYCARBONYL}-PYRROLIDINIUM; BIS-TRIFLUORO-ACETATE;
4-[[2-(1-ISOBUTYRYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM; CHLORIDE;
3-[(4-METHYL-CYCLOHEXANECARBONYL)-(1-METHYL-PIPERIDIN-4-YL)-AMINO]-5-PHENYL-THIOPHENE-2-CARBOXYLIC ACID PYRIDIN-2-YL ESTER;
4-[[2-(1-ACETOXY-1-METHYL-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
3-[(4-METHYL-CYCLOHEXANECARBONYL)-(1-METHYL-PIPERIDIN-4-YL)-AMINO]-5-PHENYL-THIOPHENE-2-CARBOXYLIC ACID 2-OXO-PYRROLIDIN-1-YLMETHYL ESTER;
1-METHYL-4-[(4-METHYL-CYCLOHEXANECARBONYL)-(2-PHENOXYCARBONYL-5-PHENYL-THIOPHEN-3-YL)-AMINO]-PIPERIDINIUM CHLORIDE;
1-METHYL-4-{(4-METHYL-CYCLOHEXANECARBONYL)-[5-PHENYL-2-(PYRIDIN-3-YLOXYCARBONYL)-THIOPHEN-3-YL]-AMINO)-PIPERIDINIUM; CHLORIDE;
4-[{2-[1-(4-HYDROXY-5-HYDROXYMETHYL-TETRAHYDRO-FURAN-2-YL)-2-OXO-1,2-DIHYDRO-PYRIMIDIN-4-YLCARBAMOYL]-5-PHENYL-THIOPHEN-3-YL}-(4-METHYL-CYCLOHEXANECARBONYL)-AMINO]-1-METHYL-PIPERIDINIUM CHLORIDE;
4-[[2-(1-METHOXYCARBONYL-2-METHYL-PROPYLCARBAMOYL)-5-PHENYL-THIOPHEN-3-YL]-(4-METHYL-CYCLOHEXANECARBONYL)-AMINO]-1-METHYL-PIPERIDINIUM CHLORIDE;
4-[[2-(1-METHOXYCARBONYL-ETHYLCARBAMOYL)-5-PHENYL-THIOPHEN-3-YL]-(4-METHYL-CYCLOHEXANECARBONYL)-AMINO]-1-METHYL-PIPERIDINIUM;
or pharmaceutically acceptable salts thereof;
and at least one pharmaceutically acceptable carrier or excipient.

19. A method for inhibiting or reducing the activity of viral polymerase in a host comprising administering to the host a therapeutically effective amount of at least one compound of formula I: 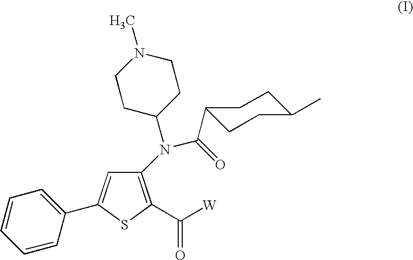
or pharmaceutically acceptable salts thereof;
wherein;
W is C₁₋₁₂ alkyloxy, C₆₋₁₂ arylalkyloxy, amino acid ester, nucleoside, C₆₋₁₂ heteroaralkyloxy, C₆ aryloxy, 5-6 membered heteroaryloxy, 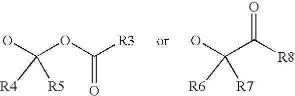
R₃ and R₈ are each independently chosen from C₁₋₁₂ alkyl, C₆₋₁₀ aryl, C₆₋₁₀ arylalkyl, C₃₋₁₀ heterocycle, C₃₋₁₂ heteroaralkyl, C₆₋₁₂ aralkyl, C₁₋₁₂ alkyloxy, C₆₋₁₀ aryloxy or NR₁₀R₁₁ wherein
R₁₀ and R₁₁ are each independently chosen from H, C₁₋₁₂ alkyl, C₆₋₁₂ aryl, C₃₋₁₀ heterocycle, C₆₋₁₂ aralkyl or C₃₋₁₀ heteroaralkyl;
R₄ and R₅ are each independently chosen from H, C₁₋₁₂ alkyl, C₆₋₁₀ aryl, —O(CO)C₁₋₆ alkyl or C₃₋₁₀ heterocycle;
R₆ and R₇ are each independently chosen from H, C₁₋₁₂ alkyl, C₆₋₁₀ aryl, —O(CO)C₁₋₆ alkyl or C₃₋₁₀ heterocycle and at least one pharmaceutically acceptable carrier or excipient.

20. A method of claim 19, wherein viral polymerase is a Flaviviridae viral polymerase.

21. A method for inhibiting or reducing the activity of viral polymerase in a host comprising administering a therapeutically effective amount of at least one compound selected from:
3-[(4-METHYL-CYCLOHEXANECARBONYL)-(1-METHYL-PIPERIDIN-4-YL)-AMINO]-5-PHENYL-THIOPHENE-2-CARBOXYLIC ACID 2,2-DIMETHYL-PROPIONYLOXYMETHY;
4-[(2-ISOPROPOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM; CHLORIDE
4-[(2-ISOPROPYLCARBAMOYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
1-METHYL-4-{(4-METHYL-CYCLOHEXANECARBONYL)-[2-(5-METHYL-2-OXO-[1,3]DIOXOL-4-YLMETHOXYCARBONYL)-5-PHENYL-THIOPHEN-3-YL]-AMINO}-PIPERIDINIUM;
4-[[2-(1-ISOPROPOXYCARBONYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
4-[{2-[1-(2,2-DIMETHYL-PROPIONYLOXY)-ETHOXYCARBONYL]-5-PHENYL-THIOPHEN-3-YL}-(4-METHYL-CYCLOHEXANECARBONYL)-AMINO]-1-METHYL-PIPERIDINIUM CHLORIDE;
4-[[2-(ISOPROPOXYCARBONYLOXY-PHENYL-METHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
4-[(2-CYCLOHEXYLOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
4-[{2-[(2,2-DIMETHYL-PROPIONYLOXY)-PHENYL-METHOXYCARBONYL]-5-PHENYL-THIOPHEN-3-YL}-(4-METHYL-CYCLOHEXANECARBONYL)-AMINO]-1-METHYL-PIPERIDINIUM CHLORIDE;
1-METHYL-4-[(4-METHYL-CYCLOHEXANECARBONYL)-(5-PHENYL-2-PROPIONYLOXYMETHOXYCARBONYL-THIOPHEN-3-YL)-AMINO]-PIPERIDINIUM; CHLORIDE;
4-[[2-(FURAN-2-CARBONYLOXYMETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
4-[(2-BENZOYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
4-[(2-CYCLOHEXANECARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
1-METHYL-4-[(4-METHYL-CYCLOHEXANECARBONYL)-(5-PHENYL-2-SUCCINYL-17(3-TERT-BUTOXYCARBONYLMETHYL-CARBAMOYL)-METHYL-PROPYL)-7,12-DIHYDROXY-10,13-DIMETHYL-HEXADECAHYDRO-CYCLOPENTA(A)PHENANTHREN-3-YLOYMETHOXYCARBONYL-THIOPHEN-3-YL)AMINO-PIPERIDINIUM CHLORIDE;
METHYL-4-[(4-METHYL-CYCLOHEXANECARBONYL)-(5-PHENYL-2-SUCCINYL-17(3-CARBONYLMETHYL-CARBAMOYL)-METHYL-PROPYL)-7,12-DIHYDROXY-10,13-DIMETHYL-HEXADECAHYDRO-CYCLOPENTA(A)PHENANTHREN-3-YLOYMETHOXYCARBONYL-THIOPHEN-3-YL)AMINO-PIPERIDINIUM CHLORIDE;
4-[(2-ETHOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
1-METHYL-4-[(4-METHYL-CYCLOHEXANECARBONYL)-(2-PHENOXYCARBONYLOXYMETHOXYCARBONYL-5-PHENYL-THIOPHEN-3-YL)-AMINO]-PIPERIDINIUM CHLORIDE;
1-METHYL-4-{(4-METHYL-CYCLOHEXANECARBONYL)-[2-(MORPHOLINE-4-CARBONYLOXYMETHOXYCARBONYL)-5-PHENYL-THIOPHEN-3-YL]-AMINO}-PIPERIDINIUM CHLORIDE;
4-[{2-[1-(2,2-DIMETHYL-PROPIONYLOXY)-2-METHYL-PROPOXYCARBONYL]-5-PHENYL-THIOPHEN-3-YL}-(4-METHYL-CYCLOHEXANECARBONYL)-AMINO]-1-METHYL-PIPERIDINIUM CHLORIDE;
4-[(2-SEC-BUTOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM CHLORIDE;
4-[[2-(1-ISOPROPOXYCARBONYLOXY-2-METHYL-PROPOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
4-[(2-SEC-BUTOXYCARBONYLOXYMETHOXYCARBONYL-5-PHENYL-THIOPHEN-3-YL)-(4-METHYL-CYCLOHEXANECARBONYL)-AMINO]-1-METHYL-PIPERIDINIUM; CHLORIDE;
3-[(4-METHYL-CYCLOHEXANECARBONYL)-(1-METHYL-PIPERIDIN-4-YL)-AMINO]-5-PHENYL-THIOPHENE-2-CARBOXYLIC ACID TERT-BUTOXYCARBONYLAMINOACETOXYMETHYL ESTER;
3-[(4-METHYL-CYCLOHEXANECARBONYL)-(1-METHYL-PIPERIDIN-4-YL)-AMINO]-5-PHENYL-THIOPHENE-2-CARBOXYLIC ACID 2-TERT-BUTOXYCARBONYLAMINO-3-METHYL-BUTYRYLOXYMETHYL ESTER;
3-[(4-METHYL-CYCLOHEXANECARBONYL)-(1-METHYL-PIPERIDIN-4-YL)-AMINO]-5-PHENYL-THIOPHENE-2-CARBOXYLIC ACID AMINOACETOXYMETHYL ESTER, BIS TRIFLUOROACETATE SALT;
3-[(4-METHYL-CYCLOHEXANECARBONYL)-(1-METHYL-PIPERIDIN-4-YL)-AMINO]-5-PHENYL-THIOPHENE-2-CARBOXYLIC ACID 2-AMINO-3-METHYL-BUTYRYLOXYMETHYL ESTER, BIS TRIFLUOROACETATE SALT;
4-[[2-(1-ISOPROPOXYCARBONYLOXY-1-METHYL-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
3-[(4-METHYL-CYCLOHEXANECARBONYL)-(1-METHYL-PIPERIDIN-4-YL)-AMINO]-5-PHENYL-THIOPHENE-2-CARBOXYLIC ACID 1-(1-METHYL-CYCLOHEXANECARBONYLOXY)-ETHYL ESTER;
4-[[2-(1-#TERT!-BUTOXYCARBONYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
1-METHYL-4-((4-METHYL-CYCLOHEXANECARBONYL)-{2-[1-(1-METHYL-CYCLOHEXANECARBONYLOXY)-ETHOXYCARBONYL]-5-PHENYL-THIOPHEN-3-YL}-AMINO)-PIPERIDINIUM;
PYRROLIDINE-1,2-DICARBOXYLIC ACID 1-TERT-BUTYL ESTER 2-{3-[(4-METHYL-CYCLOHEXANECARBONYL)-(1-METHYL-PIPERIDIN-4-YL)-AMINO]-5-PHENYL-THIOPHENE-2-CARBONYLOXYMETHYL} ESTER;
4-Methyl-piperazine-1-carboxylic acid 3-[(4-methyl-cyclohexanecarbonyl)-(1-methyl-piperidin-4-yl)-amino]-5-phenyl-thiophene-2-carbonyloxymethyl ester dihydrochloride salt;
4-[[2-(1-CYCLOHEXYLOXYCARBONYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM; CHLORIDE;
2-{3-[(4-METHYL-CYCLOHEXANECARBONYL)-(1-METHYL-PIPERIDIN-4-YL)-AMINO]-5-PHENYL-THIOPHENE-2-CARBONYLOXYMETHOXYCARBONYL}-PYRROLIDINIUM; BIS-TRIFLUORO-ACETATE;
4-[[2-(1-ISOBUTYRYLOXY-ETHOXYCARBONYL)-5-PHENYL-THIOPHEN-3-YL]-(4-METHYL-CYCLOHEXANECARBONYL)-AMINO]-1-METHYL-PIPERIDINIUM; CHLORIDE;
3-[(4-METHYL-CYCLOHEXANECARBONYL)-(1-METHYL-PIPERIDIN-4-YL)-AMINO]-5-PHENYL-THIOPHENE-2-CARBOXYLIC ACID PYRIDIN-2-YL ESTER;
4-[[2-(1-ACETOXY-1-METHYL-ETHOXYCARBONYL)-5-PHENYL-THIOPHEN-3-YL]-(4-METHYL-CYCLOHEXANECARBONYL)-AMINO]-1-METHYL-PIPERIDINIUM CHLORIDE;
3-[(4-METHYL-CYCLOHEXANECARBONYL)-(1-METHYL-PIPERIDIN-4-YL)-AMINO]-5-PHENYL-THIOPHENE-2-CARBOXYLIC ACID 2-OXO-PYRROLIDIN-1-YLMETHYL ESTER;
1-METHYL-4-[(4-METHYL-CYCLOHEXANECARBONYL)-(2-PHENOXYCARBONYL-5-PHENYL-THIOPHEN-3-YL)-AMINO]-PIPERIDINIUM CHLORIDE;
1-METHYL-4-{(4-METHYL-CYCLOHEXANECARBONYL)-[5-PHENYL-2-(PYRIDIN-3-YLOXYCARBONYL)-THIOPHEN-3-YL]-AMINO)-PIPERIDINIUM; CHLORIDE;
4-[{2-[1-(4-HYDROXY-5-HYDROXYMETHYL-TETRAHYDRO-FURAN-2-YL)-2-OXO-1,2-DIHYDRO-PYRIMIDIN-4-YLCARBAMOYL]-5-PHENYL-THIOPHEN-3-YL}-(4-METHYL-CYCLOHEXANECARBONYL)-AMINO]-1-METHYL-PIPERIDINIUM CHLORIDE;
4-[[2-(1-METHOXYCARBONYL-2-METHYL-PROPYLCARBAMOYL)-5-PHENYL-THIOPHEN-3-YL]-(4-METHYL-CYCLOHEXANECARBONYL)-AMINO]-1-METHYL-PIPERIDINIUM CHLORIDE;
4-[[2-(1-METHOXYCARBONYL-ETHYLCARBAMOYL)-5-PHENYL-THIOPHEN-3-YL]-(4-METHYL-CYCLOHEXANECARBONYL)-AMINO]-1-METHYL-PIPERIDINIUM;
or pharmaceutically acceptable salts thereof.

22. A combination comprising a least one compound of formula I: 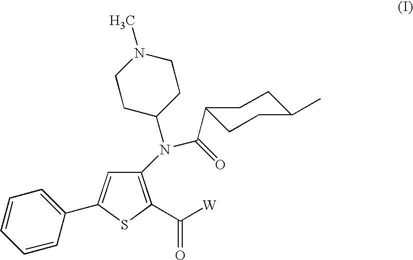
or pharmaceutically acceptable salts thereof;
wherein;
W is C₁₋₁₂ alkyloxy, C₆₋₁₂ arylalkyloxy, amino acid ester, nucleoside, C₆₋₁₂ heteroaralkyloxy, C₆ aryloxy, 5-6 membered heteroaryloxy, 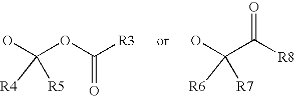
R₃ and R₈ are each independently chosen from C₁₋₁₂ alkyl, C₆₋₁₀ aryl, C₆₋₁₀ arylalkyl, C₃₋₁₀ heterocycle, C₃₋₁₂ heteroaralkyl, C₆₋₁₂ aralkyl, C₁₋₁₂ alkyloxy, C₆₋₁₀ aryloxy or NR₁₀R₁₁ wherein
R₁₀ and R₁₁ are each independently chosen from H, C₁₋₁₂ alkyl, C₆₋₁₂ aryl, C₃₋₁₀ heterocycle, C₆₋₁₂ aralkyl or C₃₋₁₀ heteroaralkyl;
R₄ and R₅ are each independently chosen from H, C₁₋₁₂ alkyl, C₆₋₁₀ aryl, —O(CO)C₁₋₆ alkyl or C₃₋₁₀ heterocycle;
R₆ and R₇ are each independently chosen from H, C₁₋₁₂ alkyl, C₆₋₁₀ aryl, —O(CO)C₁₋₆ alkyl or C₃₋₁₀ heterocycle;
and one or more additional agent chosen from viral serine protease inhibitor, viral polymerase inhibitor and viral helicase inhibitor, immunomudulating agent, antioxydant agent, antibacterial agent, therapeutic vaccine, hepatoprotectant agent or antisense agent.
